(19) United States
(12) Patent Application Publication
(10) Pub. No.: US 2007/0077283 A1
(43) Pub. Date: Apr. 5, 2007

(54) METHOD OF ENHANCING TRANSMUCOSAL DELIVERY OF THERAPEUTIC COMPOUNDS

(75) Inventors: Steven C. Quay, Seattle, WA (US); Shu-Chih Chen Quay, Seattle, WA (US); Najib Lamharzi, Bothell, WA (US); Kristine T. Fry, Seattle, WA (US)

Correspondence Address: NASTECH PHARMACEUTICAL COMPANY INC, 3830 MONTE VILLA PARKWAY, BOTHELL, WA, 98021-7266, US

(73) Assignee: Nastech Pharmaceutical Company Inc.

(21) Appl. No.: 11/536,937

(22) Filed: Sep. 29, 2006

Related U.S. Application Data
(60) Provisional application No. 60/722,334, filed on Sep. 30, 2005.
(60) Provisional application No. 60/760,815, filed on Jan. 20, 2006.
(60) Provisional application No. 60/772,311, filed on Feb. 10, 2006.

(51) Int. Cl. A61K 38/21; A61K 38/28; A61K 38/22; A61F 13/02

(52) U.S. Cl. 424/448; 514/12; 514/3; 514/9; 424/85.6; 424/85.5; 424/85.7

(57) ABSTRACT

A composition comprising a biologically active agent and a permeation enhancing lipid wherein the permeation enhancing lipid is a platelet activating factor antagonist or a biologically inactive a platelet activating factor, and increases permeability of the biologically active agent across a tissue layer. Also disclosed is a process of increasing the permeability of a biological agent across a layer tissue comprising contacting the tissue layer with a composition comprising the biological agent and a permeation enhancing lipid wherein the permeation enhancing lipid is a platelet activating factor antagonist or a biologically inactive platelet activating factor.

This patent application claims priority under 35 U.S. §119(e) of U.S. Provisional Application No. 60/722,334 filed Sep. 30, 2005, U.S. Provisional Application No. 60/760,815 filed Jan. 20, 2006, and U.S. Provisional Application No. 60/772,311 filed Feb. 10, 2006, the contents of which are incorporated herein by reference.

### BACKGROUND OF THE INVENTION

A fundamental concern in the treatment of any disease or condition is ensuring the safe and effective delivery of a therapeutic agent drug to the patient. Traditional routes of delivery for therapeutic agents include intravenous injection and oral administration. However, these delivery methods suffer from several disadvantages and thus alternative delivery systems are needed to overcome these shortcomings.

A major disadvantage of drug administration by injection is that trained personnel are often required to administer the drug. Additionally, trained personal are put in harms way when administering a drug by injection. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

The oral administration of certain therapeutic agents exhibit very low bioavailability and considerable time delay in action when given by this route due to hepatic first-pass metabolism and degradation in the gastrointestinal tract.

Mucosal administration of therapeutic compounds offers certain advantages over injection and other modes of administration, for example in terms of convenience and speed of delivery, as well as by reducing or eliminating compliance problems and side effects that attend delivery. However, mucosal delivery of biologically active agents is limited by mucosal barrier functions and other factors. Epithelial cells make up this mucosal barrier and provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. Tight junctions (TJ) of epithelial and endothelial cells are particularly important for cell-cell junctions that regulate permeability of the paracellular pathway, and also divide the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

In the context of drug delivery, the ability of drugs to permeate epithelial cell layers of mucosal surfaces, unassisted by delivery-enhancing agents, appears to be related to a number of factors, including molecular size, lipid solubility, and ionization. In general, small molecules, less than about 300-1,000 daltons, are often capable of penetrating mucosal barriers, however, as molecular size increases, permeability decreases rapidly. For these reasons, mucosal drug administration typically requires larger amounts of drug than administration by injection. Other therapeutic compounds, including large molecule drugs, are often refractory to mucosal delivery. In addition to poor intrinsic permeability, large macromolecular drugs are often subject to limited diffusion, as well as lumenal and cellular enzymatic degradation and rapid clearance at mucosal sites. Thus, in order to deliver these larger molecules in therapeutically effective amounts, cell permeation enhancing agents are required to aid their passage across these mucosal surfaces and into systemic circulation where they may quickly act on the target tissue. Therefore, there is a long-standing unmet need in the art for pharmaceutical formulations and methods of administering therapeutic compounds that are stable, well tolerated and provide enhanced mucosal delivery for a spectrum of targeted cell types including those found in the nervous system and cardiovascular system for the treatment of diseases and other adverse conditions in mammalian subjects. A related need exists for methods and compositions that will provide efficient delivery of drugs via one or more mucosal routes in therapeutic amounts, which are fast acting, easily administered and have limited adverse side effects such as mucosal irritation or tissue damage.

### SUMMARY OF THE INVENTION

One aspect of the invention is a composition comprising a biologically active agent and a permeation enhancing lipid, wherein the permeation enhancing lipid is a platelet activating factor antagonist or a biologically inactive a platelet activating factor, and and increases permeability of the biologically active agent across a tissue layer. In one embodiment of the invention, the permeation enhancing lipid is selected from the group consisting of 1-O-alkyl-2-hydroxy-sn-glycero-3-phosphocholine, 3-O-alkyl-2-acetoyl-sn-glycero-1-phosphocholine and 1-O-alkyl-2-O-acetyl-sn-glycero-3-phospho(N,N,N-trimethyl)hexanolamine. In a related embodiment of the invention, the lipid is comprised of a (C₈-C₂₂)alkyl. In another embodiment of the invention, the permeation enhancing lipid is selected from the group consisting of 1-O-hexadecyl-2-hydroxy-sn-glycero-3-phosphocholine; 1-O-octadecyl-2-hydroxy-sn-glycero-3-phosphocholine; 3-O-hexadecyl-2-acetoyl-sn-glycero-1-phosphocholine and 1-O-hexadecyl-2-O-acetyl-sn-glycero-3-phospho(N,N,N-trimethyl)hexanolamine. In yet another embodiment of the invention, the tissue layer is consists of mucosal tissue. In a related embodiment of the invention, the mucosal tissue is comprised of epithelial cells. In another related embodiment of the invention, the epithelial cell is selected from the group consisting of tracheal, bronchial, alveolar, nasal, pulmonary, gastrointestinal, epidermal or buccal. In an embodiment of the invention, the biologically active agent is a peptide or protein. In a related embodiment of the invention, the biologically active agent is preferably between about 1 kiloDalton and about 50 kiloDaltons, more preferably between about 3 kiloDaltons to about 40 kiloDaltons. In yet another related embodiment of the invention, the peptide or protein is selected from the groups consisting of peptide YY (PYY), parathyroid hormone (PTH), interferon-alpha (INF-α), interferon-beta (INF-β), interferon-gamma (INF-γ), human growth hormone (hGH), exenatide, glucagon-like peptide-1 (GLP-1), glucagon-like peptide-2 (GLP-2), glucagon-like peptide-1 derivatives, oxytocin, insulin and carbetocin. In an embodiment of the invention, the composition is further comprised of at least two poloyls. In a related embodiment of the invention, the poloyls are lactose and sorbitol. In an embodiment of the invention, the composition is further comprised of a chelating agent. In a related embodiment of the invention, the chelating agent is diamine tetraacetic acid (EDTA). In another embodiment of the invention, the composition is aqueous or solid

Another aspect of the invention is a process of increasing the permeability of a biological agent across a tissue layer comprising contacting the tissue layer with a composition comprising the biological agent and a permeation enhancing lipid, wherein the permeation enhancing lipid is a platelet activating factor antagonist or a biologically inactive platelet activating factor. In one embodiment of the invention, the permeation enhancing lipid is selected from the group consisting of 1-O-alkyl-2-hydroxy-sn-glycero-3-phosphocholine, 3-O-alkyl-2-acetoyl-sn-glycero-1-phosphocholine and 1-O-alkyl-2-O-acetyl-sn-glycero-3-phospho(N,N,N-trimethyl)hexanolamine. In a related embodiment of the invention, the lipid is comprised of a (C₈-C₂₂)alkyl. In another embodiment of the invention, the permeation enhancing lipid is selected from the group consisting of 1-O-hexadecyl-2-hydroxy-sn-glycero-3-phosphocholine; 1-O-octadecyl-2-hydroxy-sn-glycero-3-phosphocholine; 3-O-hexadecyl-2-acetoyl-sn-glycero-1-phosphocholine and 1-O-hexadecyl-2-O-acetyl-sn-glycero-3-phospho(N,N,N-trimethyl)hexanolamine. In an embodiment of the invention, the tissue layer consists of mucosal tissue. In yet another related embodiment of the invention, the mucosal tissue is comprised of epithelial cells. In a related embodiment of the invention, the epithelial cell is selected from the group consisting of tracheal, bronchial, alveolar, nasal, pulmonary, gastrointestinal, epidermal or buccal. In an embodiment of the invention, the biologically active agent is a peptide or protein. In a related embodiment of the invention, the biologically active agent is preferably between about 1 kiloDalton and about 50 kiloDaltons, more preferably between about 3 kiloDaltons to about 40 kiloDaltons. In yet another related embodiment of the invention, the peptide or protein is selected from the groups consisting of peptide YY (PYY), parathyroid hormone (PTH), interferon-alpha (INF-α), interferon-beta (INF-β), interferon-gamma (INF-γ), human growth hormone (hGH), exenatide, glucagon-like peptide-1 (GLP-1), glucagon-like peptide-2 (GLP-2), glucagon-like peptide-1 derivatives, oxytocin, insulin and carbetocin. In an embodiment of the invention, the composition is further comprised of at least two poloyls. In a related embodiment of the invention, the poloyls are lactose and sorbitol. In an embodiment of the invention, the composition is further comprised of a chelating agent. In a related embodiment of the invention, the chelating agent is diamine tetraacetic acid (EDTA). In another embodiment of the invention, the composition is aqueous or solid.

### DETAILED DESCRIPTION OF INVENTION

Abbreviations and Terms

The following abbreviations are used herein: TER, transepithelial electrical resistance; LDH, lactate dehydrogenase; MTT, tetrazolium salt; TJ, tight junction

A used herein, the term “permeation enhancing lipid” is synonymous with “tight junction modulating lipid.” Tight junction modulating lipids or TJMLs are lipids capable of compromising the integrity of the tight junctions of an epithelia with the effect of creating “openings” between epithelial cells, thus reducing the barrier function of the epithelia. Compromising the barrier function of an epithelia permits the passage of molecules, biological agents, and/or compounds across that epithelia. Permeation enhancing or TJMLS as used herein relates to a lipid that increases the amount and/or rate of delivery of a compound that is delivered into and across one or more layers of an epithelial tissue. An enhancement of delivery can be observed by measuring the rate and/or amount of the compound that passes through one or more layers of animal or human skin or other tissue. Delivery enhancement also can involve an increase in the depth into the tissue to which the compound is delivered, and/or the extent of delivery to one or more cell types including epithelial cells (e.g., tracheal, bronchial, alveolar, nasal, pulmonary, gastrointestinal, epidermal or buccal) or other tissue (e.g., increased delivery to fibroblasts, immune cells or other tissue). Permeation includes both transcellular and paracelluar transport.

The term “alkyl,” by itself or as part of another substituent, means, unless otherwise stated, a straight or branched chain, or cyclic hydrocarbon radical, or combination thereof, which may be fully saturated, mono- or polyunsaturated and can include di- and multivalent radicals, having the number of carbon atoms designated (i.e. (C₁-C₁₀) means one to ten carbons). Examples of saturated hydrocarbon radicals include groups such as methyl, ethyl, n-propyl, isopropyl, n-butyl, t-butyl, isobutyl, sec-butyl, cyclohexyl, (cyclohexyl)ethyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkylene” by itself or as part of another substituent means a divalent radical derived from an alkane, as exemplified by —CH₂CH₂CH₂CH₂—. Typically, an alkyl or alkylene group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “sugar unit” as used herein relates to a monosaccharide or it can relate to a polysaccharide. Examples of monosaccharides for use within the invention include, but are not limited to the D- and L-chiral forms of: arabinose, allose, altrose, erythrose, threose, galactose, glucose, gulose, fructose, idose, lyxose, mannose, ribose, threose, ribulose, tagatose, talose, 2-deoxyribose, and xylose. Examples of polysaccharides for use within the invention include, but are not limited to any combination of two or more monosaccharides.

General

An embodiment of the present invention provides a composition comprising a biologically active agent and a permeation enhancing lipid for the purpose of increasing the permeability of the biologically active agent across a mucosal tissue barrier, for example intranasal tissue.

Permeation enhancing lipids for use within the invention include natural or synthetic lipids and chemically modified derivatives. Thus, as used herein, the term “permeation enhancing lipid” will often be intended to embrace all of these analogs and chemically modified derivatives. In the case of lipids having carbohydrate chains or protein side chains, biologically active variants marked by alterations in these carbohydrate species are also included within the invention.

The permeation enhancing lipids and analogs for use within the methods and compositions of the invention are often formulated in a pharmaceutical composition comprising a mucosal delivery-enhancing or permeabilizing effective amount of the permeation enhancing lipid that reversibly enhances mucosal epithelial paracellular transport by modulating epithelial junctional structure and/or physiology in a mammalian subject.

Epithelial Cell Biology

Epithelial cells provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. The tight junction (TJ) of epithelial and endothelial cells is a particularly important cell-cell junction that regulates permeability of the paracellular pathway, and also divides the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration of leukocytes to reach inflammatory sites. In response to chemoattractants, leukocytes emigrate from the blood by crossing the endothelium and, in the case of mucosal infections, cross the inflamed epithelium. Transmigration occurs primarily along the paracellular rout and appears to be regulated via opening and closing of tight junctions in a highly coordinated and reversible manner.

Numerous proteins have been identified in association with TJs, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other TJ-associated molecules designated as occluding, claudins, and zonulin.

JAMs, occludin, and claudin extend into the paracellular space, and these proteins in particular have been contemplated as candidates for creating an epithelial barrier between adjacent epithelial cells and regulatable channels through epithelial cell layers. In one model, occludin, claudin, and JAM have been proposed to interact as homophilic binding partners to create a regulated barrier to paracellular movement of water, solutes, and immune cells between epithelial cells.

A cDNA encoding murine junctional adhesion molecule-1 (JAM-1) has been cloned and corresponds to a predicted type I transmembrane protein (comprising a single transmembrane domain) with a molecular weight of approximately 32-kD [Williams, et al., Molecular Immunology 36:1175-1188, 1999; Gupta, et al., IUBMB Life 50:51-56,2000; Ozaki, et al., J. Immunol 163:553-557, 1999; Martin-Padura, et al., J. Cell Biol 142:117-127, 1998]. The extracellular segment of the molecule comprises two Ig-like domains described as an amino terminal “VH-type” and a carboxy-terminal “C2-type” carboxy-terminal β-sandwich fold [Bazzoni et al., Microcirculation 8:143-152, 2001].

Another proposed trans-membrane adhesive protein involved in epithelial tight junction regulation is Occludin. Occludin is an approximately 65-kD type II transmembrane protein composed of four transmembrane domains, two extracellular loops, and a large C-terminal cytosolic domain [Furuse, et al., J. Cell Biol. 123:1777-1788, 1993; Furuse, et al., J. Cell Biol 127:1617-1626 (1994)]. This topology has been confirmed by antibody accessibility studies [Van Itallie, and Anderson, J. Cell. Sci. 110:1113-1121, 1997].

Other cytoplasmic proteins that have been localized to epithelial junctions include zonulin, symplekin, cingulin, and 7H6. Zonulins reportedly are cytoplasmic proteins that bind the cytoplasmic tail of occludin. Representing this family of proteins are “ZO-1, ZO-2, and ZO-3”. Zonulin is postulated to be a human protein analogue of the Vibrio cholerae derived zonula occludens toxin (ZOT).

Zonulin likely plays a role in tight junction regulation during developmental, physiological, and pathological processes—including tissue morphogenesis, movement of fluid, macromolecules and leukocytes between the intestinal lumen and the interstitium, and inflammatory/autoimmune disorders. See, e.g., Wang, et al., J. Cell Sci. 113:4435-40, 2000; Fasano, et al., Lancet 355:1518-9, 2000; Fasano, Ann. N.Y. Acad. Sci. 915:214-222, 2000. Zonulin expression increased in intestinal tissues during the acute phase of coeliac disease, a clinical condition in which tight junctions are opened and permeability is increased. Zonulin induces tight junction disassembly and a subsequent increase in intestinal permeability in non-human primate intestinal epithelia in vitro.

Comparison of amino acids in the active V. cholerae ZOT fragment and human zonulin identified a putative receptor binding domain within the N-terminal region of the two proteins. The ZOT biologically active domain increases intestinal permeability by interacting with a mammalian cell receptor with subsequent activation of intracellular signaling leading to the disassembly of the intercellular tight junction. The ZOT biologically active domain has been localized toward the carboxyl terminus of the protein and coincides with the predicted cleavage product generated by V. cholerae. This domain shares a putative receptor-binding motif with zonulin, the ZOT mammalian analogue. Amino acid comparison between the ZOT active fragment and zonulin, combined with site-directed mutagenesis experiments, suggest an octapeptide receptor-binding domain toward the amino terminus of processed ZOT and the amino terminus of zonulin, Di Pierro, et al., J. Biol. Chem. 276:19160-19165, 2001. ZO-1 reportedly binds actin, AF-6, ZO-associated kinase (ZAK), fodrin, and α-catenin.

Tight junction proteins are intimately associated with cell membrane lipid micrdomains called lipid rafts, which are enriched in cholesterol and glycolipids [Mrsny, R., Critical Reviews in Therapeutic Drug Carrier Systems 22(4):331-418, 2005]. Recent studies suggest that these lipid rafts act as anchors or sequestration points for the tight junction proteins claudin and occludin and may play a vital role in tight junction formation and maintenance. Claudin contains a two highly conerved domains (PQWK and GLWM) known to interact with these lipid rafts. Furthermore, occludin's transmembrane α-helix sequence is critical to this protein's ability to associate with lipid rafts within the epithelial cell membrane.

Current models of tight junction structure and function suggests that a variety of methods are available to modify tight junction integrity in order to enhance the passage of pharmaceutical formulations across epithelial cell barriers. These methods include the application of cytokines, modulation of cell-signalling components such as MAPK, modifying the phosphorylation state of tight junction proteins, down-regulating the expression of tigh junction proteins, application of small peptides homologous to domains found within tigh junction proteins that disrupt protein-protein interaction or the tight junction protein's ability to intergrate into the cell membrane and, finally, pathogen induced disruption of tight junctions [Mrsny, R., Critical Reviews in Therapeutic Drug Carrier Systems 22(4):331-418, 2005]. Although a spectrum of methods are available to modulate tight junction biology, each method has it pros and cons. For example, pathogen induced tight junction disruption has concerns regarding the safety of subjecting patients to indirect adverse effects derived from the pathogen itself. Furthermore, reversiability of compromised tight junction integrity is a key attribute to a tight junction modulator and while pathogens may be potent tight junction modulators, their reversibility is questionable. Tight junctions left in a non-reversible or a long-term “open” state leaves the patient vunerable to infection and inflammatory responses. Methods that rely on down-regulating tight protein expression are limited by a lag in response time based primarily on the half-life of the targeted tight junction protein. Lastly, there may not be a universal approach to compromise tight junction integrity based on tissue and organ specific differences in epithelia physical and chemical properties. Thus, when selecting a method to modulate tight junction integrity in order to enhance paracellular permability multiple factors must be addressed.

Platelet Activating Factor (PAF)

Platelet activating factor (PAF) refers to a lipid with the general chemical structure 1-O-alkyl-2-O-acetyl-sn-glycero-3-phorphorylcholine where the alkyl moiety is typically a 16-carbon or 18-carbon species. In its endogenous form PAF exists as a mixture of the 16-carbon and 18-carbon species. It has cell signaling function and plays a role as a mediator of inflammation, and in the mechanism of the immune response. It exerts manly different types of biological and physiological effects, including activating platelets, basophils, endothelial cells, eosinophils, lymphocytes, marcorphages, mast cells monocytes and/or neutrophils and inducing phagocytosis, exocytosis, superoxide production, chemotaxis, aggregation, proliferation, adhesion, eicosanoid generation, degranulation, calcium mobilization. The biological and physiological effects induced by PAF are mediated via G-protein coupled receptors and not their mere physical association with the cell membrane.

PAF analogs include PAF agonists, PAF antagonists and biologically inactive PAFs. PAF agonists mimick the function of PAF by mediating signaling via the same G-coupled protein receptors as PAF and exert the same biological and physiological effects as PAF. PAF antagonist may inhibit PAF signaling by blocking PAF from binding to its cell-surface receptor and/or preventing PAF from activating its cell surface receptor. A non-limiting example of a PAF antagonist is 1-O-hexadecyl-2-O-acetyl-sn-glycero-3-phospho(N,N,N-trimethyl)hexanolamine. Lastly, biologically inactive PAFs are classified as “PAFs,” but fail to induce or inhibit PAF mediated signaling. Non-limiting examples of a biologically inactive PAF include 1-O-hexadecyl-2-hydroxy-sn-glycero-3-phosphocholine; 1-O-octadecyl-2-hydroxy-sn-glycero-3-phosphocholine and 3-O-hexadecyl-2-acetoyl-sn-glycero-1-phosphocholine.

Biologically Active Agents

The methods and compositions of the present invention are directed toward enhancing mucosal, e.g., intranasal, delivery of a broad spectrum of biologically active agents to achieve therapeutic, prophylactic or other desired physiological results in mammalian subjects. As used herein, the term “biologically active agent” encompasses any substance that produces a physiological response when mucosally administered to a mammalian subject according to the methods and compositions herein. Useful biologically active agents in this context include therapeutic or prophylactic agents applied in all major fields of clinical medicine, as well as nutrients, cofactors, enzymes (endogenous or foreign), antioxidants, and the like. Thus, the biologically active agent may be water-soluble or water-insoluble, and may include higher molecular weight proteins, peptides, carbohydrates, glycoproteins, lipids, and/or glycolipids, nucleosides, polynucleotides, and other active agents.

Useful pharmaceutical agents within the methods and compositions of the invention include drugs and macromolecular therapeutic or prophylactic agents embracing a wide spectrum of compounds, including small molecule drugs, peptides, proteins, and vaccine agents. Exemplary pharmaceutical agents for use within the invention are biologically active for treatment or prophylaxis of a selected disease or condition in the subject. Biological activity in this context can be determined as any significant (i.e., measurable, statistically significant) effect on a physiological parameter, marker, or clinical symptom associated with a subject disease or condition, as evaluated by an appropriate in vitro or in vivo assay system involving actual patients, cell cultures, sample assays, or acceptable animal models.

The methods and compositions of the invention provide unexpected advantages for treatment of diseases and other conditions in mammalian subjects, which advantages are mediated, for example, by providing enhanced speed, duration, fidelity or control of mucosal delivery of therapeutic and prophylactic compounds to reach selected physiological compartments in the subject (e.g., into or across the nasal mucosa, into the systemic circulation or central nervous system (CNS), or to any selected target organ, tissue, fluid or cellular or extracellular compartment within the subject).

In various exemplary embodiments, the methods and compositions of the invention may incorporate one or more biologically active agent(s) selected from:

opioids or opioid antagonists, such as morphine, hydromorphone, oxymorphone, lovorphanol, levallorphan, codeine, nalmefene, nalorphine, nalozone, naltrexone, buprenorphine, butorphanol, and nalbufine;

corticosterones, such as cortisone, hydrocortisone, fludrocortisone, prednisone, prednisolone, methylprednisolone, triamcinolone, dexamethoasone, betamethoasone, paramethosone, and fluocinolone;

other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone;

androgens, such as testosterone;

estrogens, such as estradiol;

progestins;

muscle relaxants, such as papaverine;

vasodilators, such as nitroglycerin, vasoactive intestinal peptide and calcitonin related gene peptide;

antihistamines, such as cyproheptadine;

agents with histamine receptor site blocking activity, such as doxepin, imipramine, and cimetidine;

antitussives, such as dextromethorphan; neuroleptics such as clozaril; antiarrhythmics;

antiepileptics;

enzymes, such as superoxide dismutase and neuroenkephalinase;

anti-fungal agents, such as amphotericin B, griseofulvin, miconazole, ketoconazole, tioconazol, itraconazole, and fluconazole;

antibacterials, such as penicillins, cephalosporins, tetracyclines, aminoglucosides, erythromicin, gentamicins, polymyxin B;

anti-cancer agents, such as 5-fluorouracil, bleomycin, methotrexate, and hydroxyurea, dideoxyinosine, floxuridine, 6-mercaptopurine, doxorubicin, daunorubicin, 1-darubicin, taxol and paclitaxel (optionally provided in a bimodal emulsion, e.g., as described in U.S. patent application Ser. No. 09/631,246, filed by Quay on Aug. 2, 2000);

antioxidants, such as tocopherols, retinoids, carotenoids, ubiquinones, metal chelators, and phytic acid;

antiarrhythmic agents, such as quinidine; and

antihypertensive agents such as prazosin, verapamil, nifedipine, and diltiazem; analgesics such as acetaminophen and aspirin;

monoclonal and polyclonal antibodies, including humanized antibodies, and antibody fragments;

anti-sense oligonucleotides; and

RNA, DNA and viral vectors comprising genes encoding therapeutic peptides and proteins.

In addition to these exemplary classes and species of active agents, the methods and compositions of the invention embrace any physiologically active agent, as well as any combination of multiple active agents, described above or elsewhere herein or otherwise known in the art, that is individually or combinatorially effective within the methods and compositions of the invention for treatment or prevention of a selected disease or condition in a mammalian subject (see, Physicians' Desk Reference, published by Medical Economics Company, a division of Litton Industries, Inc).

Regardless of the class of compound employed, the biologically active agent for use within the invention will be present in the compositions and methods of the invention in an amount sufficient to provide the desired physiological effect with no significant, unacceptable toxicity or other adverse side effects to the subject. The appropriate dosage levels of all biologically active agents will be readily determined without undue experimentation by the skilled artisan. Because the methods and compositions of the invention provide for enhanced delivery of the biologically active agent(s), dosage levels significantly lower than conventional dosage levels may be used with success. In general, the active substance will be present in the composition in an amount of from about 0.01% to about 50%, often between about 0.1% to about 20%, and commonly between about 1.0% to 5% or 10% by weight of the total intranasal formulation depending upon the particular substance employed.

As used herein, the terms biolotically active “peptide” and “protein” include polypeptides of various sizes, and do not limit the invention to amino acid polymers of any particular size. Peptides from as small as a few amino acids in length, to proteins of any size, as well as peptide-peptide, protein-protein fusions and protein-peptide fusions, are encompassed by the present invention, so long as the protein or peptide is biologically active in the context of eliciting a specific physiological, immunological, therapeutic, or prophylactic effect or response.

The instant invention provides novel formulations and coordinate administration methods for enhanced mucosal delivery of biologically active peptides and proteins. Illustrative examples of therapeutic peptides and proteins for use within the invention include, but are not limited to: tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TNF), hypothalmic releasing factors, prolactin, thyroid stimulating hormone (TSH), adrenocorticotropic hormone (ACTH), parathyroid hormone (PTH), follicle stimulating hormone (FSF), luteinizing hormone releasing hormone (LHRH), endorphins, glucagon, calcitonin, oxytocin, carbetocin, aldoetecone, enkaphalins, somatostin, somatotropin, somatomedin, gonadotrophin, estrogen, progesterone, testosterone, alpha-melanocyte stimulating hormone, non-naturally occurring opiods, lidocaine, ketoprofen, sufentainil, terbutaline, droperidol, scopolamine, gonadorelin, ciclopirox, olamine, buspirone, calcitonin, cromolyn sodium or midazolam, cyclosporin, lisinopril, captopril, delapril, cimetidine, ranitidine, famotidine, superoxide dismutase, asparaginase, arginase, arginine deaminease, adenosine deaminase ribonuclease, trypsin, chemotrypsin, and papain. Additional examples of useful peptides include, but are not limited to, bombesin, substance P, vasopressin, alpha-globulins, transferrin, fibrinogen, beta-lipoproteins, beta-globulins, prothrombin, ceruloplasmin, alpha₂-glycoproteins, alpha₂-globulins, fetuin, alpha₁-lipoproteins, alpha₁-globulins, albumin, prealbumin, and other bioactive proteins and recombinant protein products.

In more detailed aspects of the invention, methods and compositions are provided for enhanced mucosal delivery of specific, biologically active peptide or protein therapeutics to treat (i.e., to eliminate, or reduce the occurrence or severity of symptoms of) an existing disease or condition, or to prevent onset of a disease or condition in a subject identified to be at risk for the subject disease or condition. Biologically active peptides and proteins that are useful within these aspects of the invention include, but are not limited to hematopoietics; antiinfective agents; antidementia agents; antiviral agents; antitumoral agents; antipyretics; analgesics; antiinflammatory agents; antiulcer agents; antiallergic agents; antidepressants; psychotropic agents; cardiotonics; antiarrythmic agents; vasodilators; antihypertensive agents such as hypotensive diuretics; antidiabetic agents; anticoagulants; cholesterol lowering agents; therapeutic agents for osteoporosis; hormones; antibiotics; vaccines; and the like.

Biologically active peptides and proteins for use within these aspects of the invention include, but are not limited to, cytokines; peptide hormones; growth factors; factors acting on the cardiovascular system; cell adhesion factors; factors acting on the central and peripheral nervous systems; factors acting on humoral electrolytes and hemal organic substances; factors acting on bone and skeleton growth or physiology; factors acting on the gastrointestinal system; factors acting on the kidney and urinary organs; factors acting on the connective tissue and skin; factors acting on the sense organs; factors acting on the immune system; factors acting on the respiratory system; factors acting on the genital organs; and various enzymes.

For example, hormones which may be administered within the methods and compositions of the present invention include androgens, estrogens, prostaglandins, somatotropins, gonadotropins, interleukins, steroids and cytokines.

Vaccines which may be administered within the methods and compositions of the present invention include bacterial and viral vaccines, such as vaccines for hepatitis, influenza, respiratory syncytial virus (RSV), parainfluenza virus (PIV), tuberculosis, canary pox, chicken pox, measles, mumps, rubella, pneumonia, and human immunodeficiency virus (HIV).

Bacterial toxoids which may be administered within the methods and compositions of the present invention include diphtheria, tetanus, pseudonomas and mycobactrium tuberculosis.

Examples of specific cardiovascular or thromobolytic agents for use within the invention include hirugen, hirulos and hirudine.

Antibody reagents that are usefully administered with the present invention include monoclonal antibodies, polyclonal antibodies, humanized antibodies, antibody fragments, fusions and multimers, and immunoglobins.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide or protein analog further includes modified forms of a native peptide or protein incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C 18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides. These CD dimers have been found to bind to hydrophobic patches of proteins in a manner that significantly inhibits aggregation. This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins [Breslow, et al., J. Am Chem. Soc. 118:11678-11681, 1996; Breslow, et al., PNAS USA 94:11156-11158, 1997].

Charge Modifying and pH Control Agents and Methods

To improve the transport characteristics of biologically active agents (e.g., macromolecular drugs, peptides or proteins) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the mucosal membrane surface, also affects permeability of the molecules. Permeation and partitioning of biologically active agents and permeabilizing agents for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

Degradative Enzyme Inhibitory Agents and Methods

A major drawback to effective mucosal delivery of biologically active agents, is that they may be subject to degradation by mucosal enzymes. The oral route of administration of therapeutic compounds is particularly problematic, because in addition to proteolysis in the stomach, the high acidity of the stomach destroys many active and inactive components of mucosal delivery formulations before they reach an intended target site of drug action. Further impairment of activity occurs by the action of gastric and pancreatic enzymes, and exo and endopeptidases in the intestinal brush border membrane, and by metabolism in the intestinal mucosa where a penetration barrier substantially blocks passage of the active agent across the mucosa.

In addition to their susceptibility to enzymatic degradation, many therapeutic compounds, particularly relatively low molecular weight proteins, and peptides, introduced into the circulation, are cleared quickly from mammalian subjects by the kidneys. This problem may be partially overcome by administering large amounts of the therapeutic compound through repeated administration. However, higher doses of therapeutic formulations containing protein or peptide components can elicit antibodies that can bind and inactivate the protein and/or facilitate the clearance of the protein from the subject's body. Repeated administration of the formulation containing the therapeutic protein or peptide is essentially ineffective and can be dangerous as it can elicit an allergic or autoimmune response.

The problem of metabolic lability of therapeutic peptides, proteins and other compounds may be addressed in part through rational drug design. However, medicinal chemists have had less success in manipulating the structures of peptides and proteins to achieve high cell membrane permeability while still retaining pharmacological activity. Unfortunately, many of the structural features of peptides and proteins (e.g., free N-terminal amino and C-terminal carboxyl groups, and side chain carboxyl (e.g., Asp, Glu), amino (e.g., Lys, Arg) and hydroxyl (e.g., Ser, Thr, Tyr) groups) that bestow upon the molecule affinity and specificity for its pharmacological binding partner also bestow upon the molecule undesirable physicochemical properties (e.g., charge, hydrogen bonding potential) which limit their cell membrane permeability. Therefore, alternative strategies need to be considered for intranasal formulation and delivery of peptide and protein therapeutics.

Exemplary mucoadhesive polymer-enzyme inhibitor complexes that are useful within the mucosal delivery formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase). See, e.g., Bemkop-Schnürch, J. Control. Rel. 52:1-16, 1998. As described in further detail below, certain embodiments of the invention will optionally incorporate a novel chitosan derivative or chemically modified form of chitosan. One such novel derivative for use within the invention is denoted as a β-[1→4]-2-guanidino-2-deoxy-D-glucose polymer (poly-GuD).

Any inhibitor that inhibits the activity of an enzyme to protect the biologically active agent(s) may be usefully employed in the compositions and methods of the invention. Useful enzyme inhibitors for the protection of biologically active proteins and peptides include, for example, soybean trypsin inhibitor, pancreatic trypsin inhibitor, chymotrypsin inhibitor and trypsin and chrymotrypsin inhibitor isolated from potato (solanum tuberosum L.) tubers. A combination or mixtures of inhibitors may be employed. Additional inhibitors of proteolytic enzymes for use within the invention include ovomucoid-enzyme, gabaxate mesylate, alpha1-antitrypsin, aprotinin, amastatin, bestatin, puromycin, bacitracin, leupepsin, alpha2-macroglobulin, pepstatin and egg white or soybean trypsin inhibitor. These and other inhibitors can be used alone or in combination. The inhibitor(s) may be incorporated in or bound to a carrier, e.g., a hydrophilic polymer, coated on the surface of the dosage form which is to contact the nasal mucosa, or incorporated in the superficial phase of said surface, in combination with the biologically active agent or in a separately administered (e.g., pre-administered) formulation.

The amount of the inhibitor, e.g., of a proteolytic enzyme inhibitor that is optionally incorporated in the compositions of the invention will vary depending on (a) the properties of the specific inhibitor, (b) the number of functional groups present in the molecule (which may be reacted to introduce ethylenic unsaturation necessary for copolymerization with hydrogel forming monomers), and (c) the number of lectin groups, such as glycosides, which are present in the inhibitor molecule. It may also depend on the specific therapeutic agent that is intended to be administered. Generally speaking, a useful amount of an enzyme inhibitor is from about 0.1 mg/ml to about 50 mg/ml, often from about 0.2 mg/ml to about 25 mg/ml, and more commonly from about 0.5 mg/ml to 5 mg/ml of the of the formulation (i.e., a separate protease inhibitor formulation or combined formulation with the inhibitor and biologically active agent).

In the case of trypsin inhibition, suitable inhibitors may be selected from, e.g., aprotinin, BBI, soybean trypsin inhibitor, chicken ovomucoid, chicken ovoinhibitor, human pancreatic trypsin inhibitor, camostat mesilate, flavonoid inhibitors, antipain, leupeptin, p-aminobenzamidine, AEBSF, TLCK (tosyllysine chloromethylketone), APMSF, DFP, PMSF, and poly(acrylate) derivatives. In the case of chymotrypsin inhibition, suitable inhibitors may be selected from, e.g., aprotinin, BBI, soybean trypsin inhibitor, chymostatin, benzyloxycarbonyl-Pro-Phe-CHO, FK-448, chicken ovoinhibitor, sugar biphenylboronic acids complexes, DFP, PMSF, β-phenylpropionate, and poly(acrylate) derivatives. In the case of elastase inhibition, suitable inhibitors may be selected from, e.g., elastatinal, methoxysuccinyl-Ala-Ala-Pro-Val-chloromethylketone (MPCMK), BBI, soybean trypsin inhibitor, chicken ovoinhibitor, DFP, and PMSF. Other naturally occurring, endogenous enzyme inhibitors for additional known degradative enzymes present in the intranasal environment, or alternatively present in preparative materials for production of intranasal formulations, will be readily ascertained by those skilled in the art for incorporation within the methods and compositions of the invention.

Among this broad group of candidate enzyme inhibitors for use within the invention are organophosphorous inhibitors, such as diisopropylfluorophosphate (DFP) and phenylmethylsulfonyl fluoride (PMSF), which are potent, irreversible inhibitors of serine proteases (e.g., trypsin and chymotrypsin). Another candidate inhibitor, 4-(2-Aminoethyl)-benzenesulfonyl fluoride (AEBSF), has an inhibitory activity comparable to DFP and PMSF, but it is markedly less toxic. (4-Aminophenyl)-methanesulfonyl fluoride hydrochloride (APMSF) is another potent inhibitor of trypsin, but is toxic in uncontrolled settings. In contrast to these inhibitors, 4-(4-isopropylpiperadinocarbonyl)phenyl1,2,3,4,-tetrahydro-1-naphthoate methanesulphonate (FK-448) is a low toxic substance, representing a potent and specific inhibitor of chymotrypsin. Further representatives of this non-protein group of inhibitor candidates, and also exhibiting low toxic risk, are camostat mesilate (N,N′-dimethyl carbamoylmethyl-p-(p′-guanidino-benzoyloxy)phenylacetate methane-sulphonate) and Na-glycocholate [Yamamoto, et al., Pharm. Res. 11:1496-1500, 1994; Okagava, et al., Life Sci. 55:677-683, 1994].

Yet another type of enzyme inhibitory agent for use within the methods and compositions of the invention are amino acids and modified amino acids that interfere with enzymatic degradation of specific therapeutic compounds. For use in this context, amino acids and modified amino acids are substantially non-toxic and can be produced at a low cost. However, due to their low molecular size and good solubility, they are readily diluted and absorbed in mucosal environments. Nevertheless, under proper conditions, amino acids can act as reversible, competitive inhibitors of protease enzymes. See, e.g., McClellan, et al., Biochim. Biophys. Acta. 613:160-167, 1980. Certain modified amino acids can display a much stronger inhibitory activity. A desired modified amino acid in this context is known as a ‘transition-state’ inhibitor. The strong inhibitory activity of these compounds is based on their structural similarity to a substrate in its transition-state geometry, while they are generally selected to have a much higher affinity for the active site of an enzyme than the substrate itself. Transition-state inhibitors are reversible, competitive inhibitors. Examples of this type of inhibitor are α-aminoboronic acid derivatives, such as boro-leucine, boro-valine and boro-alanine. The boron atom in these derivatives can form a tetrahedral boronate ion that is believed to resemble the transition state of peptides during their hydrolysis by aminopeptidases. Another modified amino acid for which a strong protease inhibitory activity has been reported is N-acetylcysteine, which inhibits enzymatic activity of aminopeptidase N. Still other useful enzyme inhibitors for use within the coordinate administration methods and combinatorial formulations of the invention may be selected from peptides and modified peptide enzyme inhibitors. An important representative of this class of inhibitors is the cyclic dodecapeptide, bacitracin, obtained from Bacillus licheniformis. Bacitracin A has a molecular mass of 1423 Da and shows remarkable resistance against the action of proteolytic enzymes like trypsin and pepsin. It has several biological properties inhibiting bacterial peptidoglycan synthesis, mammalian transglutaminase activity, and proteolytic enzymes such as aminopeptidase N.

In addition to these types of peptides, certain dipeptides and tripeptides display weak, non-specific inhibitory activity towards some proteases, Langguth, et al., J. Pharm. Pharmacol. 46:34-40, 1994. By analogy with amino acids, their inhibitory activity can be improved by chemical modifications. For example, phosphinic acid dipeptide analogues are also ‘transition-state’ inhibitors with a strong inhibitory activity towards aminopeptidases. They have reportedly been used to stabilize nasally administered leucine enkephalin, Hussain, et al., Pharm. Res. 9:626-628, 1992. Another example of a transition-state analogue is the modified pentapeptide pepstatin, which is a very potent inhibitor of pepsin. Structural analysis of pepstatin, by testing the inhibitory activity of several synthetic analogues, demonstrated the major structure-function characteristics of the molecule responsible for the inhibitory activity [McConnell, et al., J. Med. Chem. 34:2298-2300, 1991. Similar analytic methods can be readily applied to prepare modified amino acid and peptide analogs for blockade of selected, intranasal degradative enzymes.

Another special type of modified peptide includes inhibitors with a terminally located aldehyde function in their structure. For example, the sequence benzyloxycarbonyl-Pro-Phe-CHO, which fulfills the known primary and secondary specificity requirements of chymotrypsin, has been found to be a potent reversible inhibitor of this target proteinase.

Additional agents for protease inhibition within the formulations and methods of the invention involve the use of complexing agents. These agents mediate enzyme inhibition by depriving the intranasal environment (or preparative or therapeutic composition) of divalent cations which are co-factors for many proteases. For instance, the complexing agents EDTA and DTPA as coordinately administered or combinatorially formulated adjunct agents, in suitable concentration, will be sufficient to inhibit selected proteases to thereby enhance intranasal delivery of biologically active agents according to the invention. Further representatives of this class of inhibitory agents are EGTA, 1,10-phenanthroline and hydroxychinoline.

Exemplary mucoadhesive polymer-enzyme inhibitor complexes that are useful within the mucosal formulations and methods of the invention include, but are not limited to: Carboxymethylcellulose-pepstatin (with anti-pepsin activity); Poly(acrylic acid)-Bowman-Birk inhibitor (anti-chymotrypsin); Poly(acrylic acid)-chymostatin (anti-chymotrypsin); Poly(acrylic acid)-elastatinal (anti-elastase); Carboxymethylcellulose-elastatinal (anti-elastase); Polycarbophil-elastatinal (anti-elastase); Chitosan-antipain (anti-trypsin); Poly(acrylic acid)-bacitracin (anti-aminopeptidase N); Chitosan-EDTA (anti-aminopeptidase N, anti-carboxypeptidase A); Chitosan-EDTA-antipain (anti-trypsin, anti-chymotrypsin, anti-elastase).

Ciliostatic Agents and Methods

Because the self-cleaning capacity of certain mucosal tissues (e.g., nasal mucosal tissues) by mucociliary clearance is necessary as a protective function (e.g., to remove dust, allergens, and bacteria), it has been generally considered that this function should not be substantially impaired by mucosal medications. Mucociliary transport in the respiratory tract is a particularly important defense mechanism against infections. To achieve this function, ciliary beating in the nasal and airway passages moves a layer of mucus along the mucosa to removing inhaled particles and microorganisms.

Various reports show that mucociliary clearance can be impaired by mucosally administered drugs, as well as by a wide range of formulation additives including penetration enhancers and preservatives. Within more detailed aspects, a specific ciliostatic factor is employed in a combined formulation or coordinate administration protocol with one or more biologically active agents. Various bacterial ciliostatic factors isolated and characterized in the literature may be employed within these embodiments of the invention. For example, ciliostatic factors from the bacterium Pseudomonas aeruginosa have been identified, Hingley, et al., Infection and Immunity 51:254-262, 1986. These are heat-stable factors released by Pseudomonas aeruginosa in culture supernatants that have been shown to inhibit ciliary function in epithelial cell cultures. Exemplary among these cilioinhibitory components are a phenazine derivative, a pyo compound (2-alkyl-4-hydroxyquinolines), and a rhamnolipid (also known as a hemolysin). Inhibitory concentrations of these and other active components were established by quantitative measures of ciliary motility and beat frequency. The pyo compound produced ciliostasis at concentrations of 50 μg/ml and without obvious ultrastructural lesions. The phenazine derivative also inhibited ciliary motility but caused some membrane disruption, although at substantially greater concentrations of 400 μg/ml. Limited exposure of tracheal explants to the rhamnolipid resulted in ciliostasis which was associated with altered ciliary membranes. More extensive exposure to rhamnolipid was associated with removal of dynein arms from axonemes. It is proposed that these and other bacterial ciliostatic factors have evolved to enable P. aeruginosa to more easily and successfully colonize the respiratory tract of mammalian hosts. On this basis, respiratory bacteria are useful pathogens for identification of suitable, specific ciliostatic factors for use within the methods and compositions of the invention. Rhamnolipids described in Zulianello, et al., Infect. Immun. 74(6):3134-3147, 2006, are hereby incorporated by reference. The rhamnolipids disclosed therein are non-toxic tight junction modulating lipids that promote the permeation of an epithelia and may be used herein with the present invention.

Mucosal Delivery Enhancement Agents

Additional mucosal delivery-enhancing agents that are useful within the coordinate administration and processing methods and combinatorial formulations of the invention include, but are not limited to, mixed micelles; enamines; nitric oxide donors (e.g., S-nitroso-N-acetyl-DL-penicillamine, NOR1, NOR4—which are preferably co-administered with an NO scavenger such as carboxy-PITO or doclofenac sodium); sodium salicylate; glycerol esters of acetoacetic acid (e.g., glyceryl-1,3-diacetoacetate or 1,2-isopropylideneglycerine-3-acetoacetate); and other release-diffusion or intra- or trans-epithelial penetration-promoting agents that are physiologically compatible for mucosal delivery. Other absorption-promoting agents are selected from a variety of carriers, bases and excipients that enhance mucosal delivery, stability, activity or trans-epithelial penetration of the Y2 receptor-binding peptide. These include, inter alia, α, β, or γ-cyclodextrins and derivatives and especially β-cyclodextrin derivatives (e.g., 2-hydroxypropyl-β-cyclodextrin and heptakis(2,6-di-O-methyl-β-cyclodextrin) methylated cyclodextrins (methyl-β-cyclodextrin and dimethyl-β-cyclodextrin), ethylated cyclodextrins, hydroxypropylated cyclodextrins, polymeric cyclodextrins. These compounds, optionally conjugated with one or more of the active ingredients and further optionally formulated in an oleaginous base, enhance bioavailability in the mucosal formulations of the invention. Yet additional absorption-enhancing agents adapted for mucosal delivery include medium-chain fatty acids, including mono- and diglycerides (e.g., sodium caprate—extracts of coconut oil, Capmul), and triglycerides (e.g., amylodextrin, Estaram 299, Miglyol 810).

Chelating Agents

Many formulations is contain one or more chelating agent such as diethylene triamine tetraacetic acid (DTPA), ethylene diamine tetraacetic acid (EDTA) (including edetate calcium disodium, edetate disodium, and edetate trisodium), deferiprone, deferoxamine, ditiocarb sodium, penicillamine, pentetate calcium trisodium, pentetic acid, succimer, trientine or ethylene glycol tetraacetic acid (EGTA).

Tonicifying Salts

Many formulations contain tonicifying salts, which include, but are not limited to sodium acetate, sodium bicarbonate, sodium carbonate, sodium chloride, potassium acetate, potassium bicarbonate, potassium carbonate, and potassium chloride.

Preservatives

Also a preservative such as chlorobutanol, methyl paraben, propyl paraben, sodium benzoate (0.5%), phenol, cresol, p-chloro-m-cresol, phenylethyl alcohol, benzyl alcohol, phenylmercuric acetate, phenylmercuric borate, phenylmercuric nitrate, thimerosal, sorbic acid, benzethonium chloride or benzylkonium chloride can be added to the formulation to inhibit microbial growth.

The pH is generally regulated using a buffer such as a system comprised of citric acid and a citrate salt(s), such as sodium citrate. Additional suitable buffer systems include acetic acid and an acetate salt system, succinic acid and a succinate salt system, malic acid and a malic salt system, and gluconic acid and a gluconate salt system. Alternatively, buffer systems comprised of mixed acid/salt systems can be employed, such as an acetic acid and sodium citrate system, a citrate acid, sodium acetate system, and a citric acid, sodium citrate, sodium benzoate system. For any buffer system, additional acids, such as hydrochloric acid, and additional bases, such as sodium hydroxide, may be added for final pH adjustment.

Degradation Enzymes and Inhibitors of Fatty Acid and Cholesterol Synthesis

In related aspects of the invention, biologically active agents for mucosal administration are formulated or coordinately administered with a penetration enhancing agent selected from a degradation enzyme, or a metabolic stimulatory agent or inhibitor of synthesis of fatty acids, sterols or other selected epithelial barrier components (see, e.g., U.S. Pat. No. 6,190,894). In one embodiment, known enzymes that act on mucosal tissue components to enhance permeability are incorporated in a combinatorial formulation or coordinate administration method of instant invention, as processing agents within the multi-processing methods of the invention. For example, degradative enzymes such as phospholipase, hyaluronidase, neuraminidase, and chondroitinase may be employed to enhance mucosal penetration of biologically active agents without causing irreversible damage to the mucosal barrier. In one embodiment, chondroitinase is employed within a method or composition as provided herein to alter glycoprotein or glycolipid constituents of the permeability barrier of the mucosa, thereby enhancing mucosal absorption of biologically active agents.

With regard to inhibitors of synthesis of mucosal barrier constituents, it is noted that free fatty acids account for 20-25% of epithelial lipids by weight. Inhibitors of free fatty acid synthesis and metabolism for use within the methods and compositions of the invention include, but are not limited to, inhibitors of acetyl CoA carboxylase such as 5-tetradecyloxy-2-furancarboxylic acid (TOFA); inhibitors of fatty acid synthetase; inhibitors of phospholipase A such as gomisin A, 2-(p-amylcinnamyl)amino-4-chlorobenzoic acid, bromophenacyl bromide, monoalide, 7,7-dimethyl-5,8-eicosadienoic acid, nicergoline, cepharanthine, nicardipine, quercetin, dibutyryl-cyclic AMP, R-24571, N-oleoylethanolamine, N-(7-nitro-2,1,3-benzoxadiazol-4-yl) phosphostidyl serine, cyclosporine A, topical anesthetics, including dibucaine, prenylamine, retinoids, such as all-trans and 13-cis-retinoic acid, W-7, trifluoperazine, R-24571 (calmidazolium), 1-hexadocyl-3-trifluoroethyl glycero-sn-2-phosphomenthol (MJ33); calcium channel blockers including nicardipine, verapamil, diltiazem, nifedipine, and nimodipine; antimalarials including quinacrine, mepacrine, chloroquine and hydroxychloroquine; beta blockers including propanalol and labetalol; calmodulin antagonists; EGTA; thimersol; glucocorticosteroids including dexamethasone and prednisolone; and nonsteroidal antiinflammatory agents including indomethacin and naproxen.

Free sterols, primarily cholesterol, account for 20-25% of the epithelial lipids by weight. The rate limiting enzyme in the biosynthesis of cholesterol is 3-hydroxy-3-methylglutaryl (HMG) CoA reductase. Inhibitors of cholesterol synthesis for use within the methods and compositions of the invention include, but are not limited to, competitive inhibitors of (HMG) CoA reductase, such as simvastatin, lovastatin, fluindostatin (fluvastatin), pravastatin, mevastatin, as well as other HMG CoA reductase inhibitors, such as cholesterol oleate, cholesterol sulfate and phosphate, and oxygenated sterols, such as 25-OH— and 26-OH— cholesterol; inhibitors of squalene synthetase; inhibitors of squalene epoxidase; inhibitors of DELTA7 or DELTA24 reductases such as 22,25-diazacholesterol, 20,25-diazacholestenol, AY9944, and triparanol.

Each of the inhibitors of fatty acid synthesis or the sterol synthesis inhibitors may be coordinately administered or combinatorially formulated with one or more biologically active agents to achieve enhanced epithelial penetration of the active agent(s). An effective concentration range for the sterol inhibitor in a therapeutic or adjunct formulation for mucosal delivery is generally from about 0.0001% to about 20% by weight of the total, more typically from about 0.01% to about 5%.

Nitric Oxide Donor Agents and Methods

Within other related aspects of the invention, a nitric oxide (NO) donor is selected as a membrane penetration-enhancing agent to enhance mucosal delivery of one or more biologically active agents. Various NO donors are known in the art and are useful in effective concentrations within the methods and formulations of the invention. Exemplary NO donors include, but are not limited to, nitroglycerine, nitropruside, NOC5 [3-(2-hydroxy-1-(methyl-ethyl)-2-nitrosohydrazino)-1-propanamine], NOC12 [N-ethyl-2-(1-ethyl-hydroxy-2-nitrosohydrazino)-ethanamine], SNAP [S-nitroso-N-acetyl-DL-penicillamine], NORI and NOR4. Within the methods and compositions of the invention, an effective amount of a selected NO donor is coordinately administered or combinatorially formulated with one or more biologically active agents into or through the mucosal epithelium.

Additional Agents for Modulating Epithelial Junction Structure and/or Physiology

Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). See, also WO 96/37196; U.S. Pat. Nos. 5,945,510; 5,948,629; 5,912,323; 5,864,014; 5,827,534; 5,665,389; and 5,908,825. Thus, ZOT and other agents that modulate the ZO1-ZO2 complex will be combinatorially formulated or coordinately administered with one or more biologically active agents.

Vasodilator Agents and Methods

Yet another class of absorption-promoting agents that shows beneficial utility within the coordinate administration and combinatorial formulation methods and compositions of the invention are vasoactive compounds, more specifically vasodilators. These compounds function within the invention to modulate the structure and physiology of the submucosal vasculature, increasing the transport rate of biologically active agents into or through the mucosal epithelium and/or to specific target tissues or compartments.

Vasodilator agents for use within the invention typically are generally divided into 9 classes: calcium antagonists, potassium channel openers, ACE inhibitors, angiotensin-II receptor antagonists, α-adrenergic and imidazole receptor antagonists, β1-adrenergic agonists, phosphodiesterase inhibitors, eicosanoids and NO donors.

Despite chemical differences, the pharmacokinetic properties of calcium antagonists are similar. Absorption into the systemic circulation is high, and these agents therefore undergo considerable first-pass metabolism by the liver, resulting in individual variation in pharmacokinetics. Except for the newer drugs of the dihydropyridine type (amlodipine, felodipine, isradipine, nilvadipine, nisoldipine and nitrendipine), the half-life of calcium antagonists is short. Therefore, to maintain an effective drug concentration for many of these may require delivery by multiple dosing, or controlled release formulations, as described elsewhere herein. Treatment with the potassium channel opener minoxidil may also be limited in manner and level of administration due to potential adverse side effects.

ACE inhibitors prevent conversion of angiotensin-I to angiotensin-II, and are most effective when renin production is increased. Since ACE is identical to kininase-II, which inactivates the potent endogenous vasodilator bradykinin, ACE inhibition causes a reduction in bradykinin degradation. ACE inhibitors provide the added advantage of cardioprotective and cardioreparative effects, by preventing and reversing cardiac fibrosis and ventricular hypertrophy in animal models. The predominant elimination pathway of most ACE inhibitors is via renal excretion. Therefore, renal impairment is associated with reduced elimination and a dosage reduction of 25 to 50% is recommended in patients with moderate to severe renal impairment.

Selective Transport-Enhancing Agents and Methods

Exemplary selective transport-enhancing agents for use within this aspect of the invention include, but are not limited to, glycosides, sugar-containing molecules, and binding agents such as lectin binding agents, which are known to interact specifically with epithelial transport barrier components. Certain bioadhesive ligands for use within the invention will mediate transmission of biological signals to epithelial target cells that trigger selective uptake of the adhesive ligand by specialized cellular transport processes (endocytosis or transcytosis). These transport mediators can therefore be employed as a “carrier system” to stimulate or direct selective uptake of one or more biologically active agent into and/or through mucosal epithelia.

Lectins are plant proteins that bind to specific sugars found on the surface of glycoproteins and glycolipids of eukaryotic cells. Concentrated solutions of lectins have a ‘mucotractive’ effect, and various studies have demonstrated rapid receptor mediated endocytocis (RME) of lectins and lectin conjugates (e.g., concanavalin A conjugated with colloidal gold particles) across mucosal surfaces. Additional studies have reported that the uptake mechanisms for lectins can be utilized for intestinal drug targeting in vivo. In certain of these studies, polystyrene nanoparticles (500 nm) were covalently coupled to tomato lectin and reported yielded improved systemic uptake after oral administration to rats.

In addition to plant lectins, microbial adhesion and invasion factors provide a rich source of candidates for use as adhesive/selective transport carriers within the mucosal delivery methods and compositions of the invention. See, e.g., Lehr, Crit. Rev. Therap. Drug Carrier Syst. 11:177-218, 1995; Swann, P. A., Pharmaceutical Research 15:826-832, 1998. Two components are necessary for bacterial adherence processes, a bacterial ‘adhesin’ (adherence or colonization factor) and a receptor on the host cell surface.

Various plant toxins, mostly ribosome-inactivating proteins (RIPs), have been identified that bind to any mammalian cell surface expressing galactose units and are subsequently internalized by REM. Toxins such as nigrin b, α-sarcin, ricin and saporin, viscumin, and modeccin are highly toxic upon oral administration (i.e., are rapidly internalized). Therefore, modified, less toxic subunits of these compounds will be useful within the invention to facilitate the uptake of biologically active agents.

Viral haemagglutinins comprise another type of transport agent to facilitate mucosal delivery of biologically active agents within the methods and compositions of the invention. The initial step in many viral infections is the binding of surface proteins (haemagglutinins) to mucosal cells. These binding proteins have been identified for most viruses, including rotaviruses, varicella zoster virus, semliki forest virus, adenoviruses, potato leafroll virus, and reovirus. These and other exemplary viral hemagglutinins can be employed in a combinatorial formulation (e.g., a mixture or conjugate formulation) or coordinate administration protocol with one or more biologically active agent.

Polymeric Delivery Vehicles and Methods

Within certain aspects of the invention, biologically active agents, and delivery-enhancing agents as described above, are, individually or combinatorially, incorporated within a mucosally (e.g., nasally) administered formulation that includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the biologically active agent can be functionalized in a manner where it can be covalently bound to the polymer and rendered inseparable from the polymer by simple washing. In other embodiments, the polymer is chemically modified with an inhibitor of enzymes or other agents which may degrade or inactivate the biologically active agent(s) and/or delivery enhancing agent(s). In certain formulations, the polymer is a partially or completely water insoluble but water swellable polymer, e.g., a hydrogel. Polymers useful in this aspect of the invention are desirably water interactive and/or hydrophilic in nature to absorb significant quantities of water, and they often form hydrogels when placed in contact with water or aqueous media for a period of time sufficient to reach equilibrium with water. In more detailed embodiments, the polymer is a hydrogel which, when placed in contact with excess water, absorbs at least two times its weight of water at equilibrium when exposed to water at room temperature (see, e.g., U.S. Pat. No. 6,004,583).

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Biodegradable polymers such as poly(glycolic acid) (PGA), poly-(lactic acid) (PLA), and poly(D,L-lactic-co-glycolic acid) (PLGA), have received considerable attention as possible drug delivery carriers, since the degradation products of these polymers have been found to have low toxicity. Absorption-promoting polymers of the invention may include polymers from the group of homo- and copolymers based on various combinations of the following vinyl monomers: acrylic and methacrylic acids, acrylamide, methacrylamide, hydroxyethylacrylate or methacrylate, vinylpyrrolidones, as well as polyvinylalcohol and its co- and terpolymers, polyvinylacetate, its co- and terpolymers with the above listed monomers and 2-acrylamido-2-methyl-propanesulfonic acid (AMPS®). Very useful are copolymers of the above listed monomers with copolymerizable functional monomers such as acryl or methacryl amide acrylate or methacrylate esters where the ester groups are derived from straight or branched chain alkyl, aryl having up to four aromatic rings which may contain alkyl substituents of 1 to 6 carbons; steroidal, sulfates, phosphates or cationic monomers such as N,N-dimethylaminoalkyl(meth)acrylamide, dimethylaminoalkyl(meth)acrylate, (meth)acryloxyalkyltrimethylammonium chloride, (meth)acryloxyalkyldimethylbenzyl ammonium chloride.

Additional absorption-promoting polymers for use within the invention are those classified as dextrans, dextrins, and from the class of materials classified as natural gums and resins, or from the class of natural polymers such as processed collagen, chitin, chitosan, pullalan, zooglan, alginates and modified alginates such as “Kelcoloid” (a polypropylene glycol modified alginate) gellan gums such as “Kelocogel,” Xanathan gums such as “Keltrol,” estastin, alpha hydroxy butyrate and its copolymers, hyaluronic acid and its derivatives, polylactic and glycolic acids.

A very useful class of polymers applicable within the instant invention are olefinically-unsaturated carboxylic acids containing at least one activated carbon-to-carbon olefinic double bond, and at least one carboxyl group; that is, an acid or functional group readily converted to an acid containing an olefinic double bond which readily functions in polymerization because of its presence in the monomer molecule, either in the alpha-beta position with respect to a carboxyl group, or as part of a terminal methylene grouping. Olefinically-unsaturated acids of this class include such materials as the acrylic acids typified by the acrylic acid itself, alpha-cyano acrylic acid, beta methylacrylic acid (crotonic acid), alpha-phenyl acrylic acid, beta-acryloxy propionic acid, cinnamic acid, p-chloro cinnamic acid, 1-carboxy-4-phenyl butadiene-1,3, itaconic acid, citraconic acid, mesaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same carboxylic acid molecule.

Representative acrylates useful as absorption-promoting agents within the invention include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl methacrylate, nonyl acrylate, hexyl acrylate, n-hexyl methacrylate, and the like. Higher alkyl acrylic esters are decyl acrylate, isodecyl methacrylate, lauryl acrylate, stearyl acrylate, behenyl acrylate and melissyl acrylate and methacrylate versions thereof. Mixtures of two or three or more long chain acrylic esters may be successfully polymerized with one of the carboxylic monomers. Other comonomers include olefins, including alpha olefins, vinyl ethers, vinyl esters, and mixtures thereof.

Other vinylidene monomers, including the acrylic nitriles, may also be used as absorption-promoting agents within the methods and compositions of the invention to enhance delivery and absorption of one or more biologically active agent(s), including to enhance delivery of the active agent to a target tissue or compartment in the subject (e.g., the systemic circulation or CNS). Useful alpha, beta-olefinically unsaturated nitriles are preferably monoolefinically unsaturated nitriles having from 3 to 10 carbon atoms such as acrylonitrile, methacrylonitrile, and the like. Most preferred are acrylonitrile and methacrylonitrile. Acrylic amides containing from 3 to 35 carbon atoms including monoolefinically unsaturated amides also may be used. Representative amides include acrylamide, methacrylamide, N-t-butyl acrylamide, N-cyclohexyl acrylamide, higher alkyl amides, where the alkyl group on the nitrogen contains from 8 to 32 carbon atoms, acrylic amides including N-alkylol amides of alpha, beta-olefinically unsaturated carboxylic acids including those having from 4 to 10 carbon atoms such as N-methylol acrylamide, N-propanol acrylamide, N-methylol methacrylamide, N-methylol maleimide, N-methylol maleamic acid esters, N-methylol-p-vinyl benzamide, and the like.

Yet additional useful absorption promoting materials are alpha-olefins containing from 2 to 18 carbon atoms, more preferably from 2 to 8 carbon atoms; dienes containing from 4 to 10 carbon atoms; vinyl esters and allyl esters such as vinyl acetate; vinyl aromatics such as styrene, methyl styrene and chloro-styrene; vinyl and allyl ethers and ketones such as vinyl methyl ether and methyl vinyl ketone; chloroacrylates; cyanoalkyl acrylates such as alpha-cyanomethyl acrylate, and the alpha-, beta-, and gamma-cyanopropyl acrylates; alkoxyacrylates such as methoxy ethyl acrylate; haloacrylates as chloroethyl acrylate; vinyl halides and vinyl chloride, vinylidene chloride and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(beta-haloalkyl)alkenyl phosphonates such as bis(beta-chloroethyl)vinyl phosphonate and the like as are known to those skilled in the art. Copolymers wherein the carboxy containing monomer is a minor constituent, and the other vinylidene monomers present as major components are readily prepared in accordance with the methods disclosed herein.

When hydrogels are employed as absorption promoting agents within the invention, these may be composed of synthetic copolymers from the group of acrylic and methacrylic acids, acrylamide, methacrylamide, hydroxyethylacrylate (HEA) or methacrylate (HEMA), and vinylpyrrolidones which are water interactive and swellable. Specific illustrative examples of useful polymers, especially for the delivery of peptides or proteins, are the following types of polymers: (meth)acrylamide and 0.1 to 99 wt. % (meth)acrylic acid; (meth)acrylamides and 0.1-75 wt % (meth)acryloxyethyl trimethyammonium chloride; (meth)acrylamide and 0.1-75 wt % (meth)acrylamide; acrylic acid and 0.1-75 wt % alkyl(meth)acrylates; (meth)acrylamide and 0.1-75 wt % AMPS® (trademark of Lubrizol Corp.); (meth)acrylamide and 0 to 30 wt % alkyl(meth)acrylamides and 0.1-75 wt % AMPS®; (meth)acrylamide and 0.1-99 wt. % HEMA; (metb)acrylamide and 0.1 to 75 wt % HEMA and 0.1 to 99% (meth)acrylic acid; (meth)acrylic acid and 0.1-99 wt % HEMA; 50 mole % vinyl ether and 50 mole % maleic anhydride; (meth)acrylamide and 0.1 to 75 wt % (meth)acryloxyalky dimethyl benzylammonium chloride; (meth)acrylamide and 0.1 to 99 wt % vinyl pyrrolidone; (meth)acrylamide and 50 wt % vinyl pyrrolidone and 0.1-99.9 wt % (meth)acrylic acid; (meth)acrylic acid and 0.1 to 75 wt % AMPS® and 0.1-75 wt % alkyl(meth)acrylamide. In the above examples, alkyl means C₁ to C₃₀, preferably C₁ to C₂₂, linear and branched and C₄ to C₁₆ cyclic; where (meth) is used, it means that the monomers with and without the methyl group are included. Other very useful hydrogel polymers are swellable, but insoluble versions of poly(vinyl pyrrolidone) starch, carboxymethyl cellulose and polyvinyl alcohol.

Additional polymeric hydrogel materials useful within the invention include (poly)hydroxyalkyl (meth)acrylate: anionic and cationic hydrogels: poly(electrolyte) complexes; poly(vinyl alcohols) having a low acetate residual: a swellable mixture of crosslinked agar and crosslinked carboxymethyl cellulose: a swellable composition comprising methyl cellulose mixed with a sparingly crosslinked agar; a water swellable copolymer produced by a dispersion of finely divided copolymer of maleic anhydride with styrene, ethylene, propylene, or isobutylene; a water swellable polymer of N-vinyl lactams; swellable sodium salts of carboxymethyl cellulose; and the like.

Other gelable, fluid imbibing and retaining polymers useful for forming the hydrophilic hydrogel for mucosal delivery of biologically active agents within the invention include pectin; polysaccharides such as agar, acacia, karaya, tragacenth, algins and guar and their crosslinked versions; acrylic acid polymers, copolymers and salt derivatives, polyacrylamides; water swellable indene maleic anhydride polymers; starch graft copolymers; acrylate type polymers and copolymers with water absorbability of about 2 to 400 times its original weight; diesters of polyglucan; a mixture of crosslinked poly(vinyl alcohol) and poly(N-vinyl-2-pyrrolidone); polyoxybutylene-polyethylene block copolymer gels; carob gum; polyester gels; poly urea gels; polyether gels; polyamide gels; polyimide gels; polypeptide gels; polyamino acid gels; poly cellulosic gels; crosslinked indene-maleic anhydride acrylate polymers; and polysaccharides.

In more detailed aspects of the invention, mucosal delivery of biologically active agents, is enhanced by retaining the active agent(s) in a slow-release or enzymatically or physiologically protective carrier or vehicle, for example a hydrogel that shields the active agent from the action of the degradative enzymes. In certain embodiments, the active agent is bound by chemical means to the carrier or vehicle, to which may also be admixed or bound additional agents such as enzyme inhibitors, cytokines, etc. The active agent may alternately be immobilized through sufficient physical entrapment within the carrier or vehicle, e.g., a polymer matrix.

Polymers such as hydrogels useful within the invention may incorporate functional linked agents such as glycosides chemically incorporated into the polymer for enhancing intranasal bioavailability of active agents formulated therewith. Examples of such glycosides are glucosides, fructosides, galactosides, arabinosides, mannosides and their alkyl substituted derivatives and natural glycosides such as arbutin, phlorizin, amygdalin, digitonin, saponin, and indican.

Bioadhesive Delivery Vehicles and Methods:

In certain aspects of the invention, the combinatorial formulations and/or coordinate administration methods herein incorporate an effective amount of a nontoxic bioadhesive as an adjunct compound or carrier to enhance mucosal delivery of one or more biologically active agent(s). Bioadhesive agents in this context exhibit general or specific adhesion to one or more components or surfaces of the targeted mucosa. The bioadhesive maintains a desired concentration gradient of the biologically active agent into or across the mucosa to ensure penetration of even large molecules (e.g., peptides and proteins) into or through the mucosal epithelium. Typically, employment of a bioadhesive within the methods and compositions of the invention yields a two- to five- fold, often a five- to ten-fold increase in permeability for peptides and proteins into or through the mucosal epithelium.

A variety of suitable bioadhesives are disclosed in the art for oral administration. See, e.g., U.S. Pat. Nos. 3,972,995; 4,259,314; 4,680,323; 4,740,365; 4,573,996; 4,292,299; 4,715,369; 4,876,092; 4,855,142; 4,250,163; 4,226,848; 4,948,580; U.S. Pat. Reissue No. 33,093; and Robinson, 18 Proc. Intern. Symp. Control Rel. Bioact. Mater. 75, 1991.

In certain aspects of the invention, bioadhesive materials for enhancing intranasal delivery of biologically active agents comprise a matrix of a hydrophilic, e.g., water soluble or swellable, polymer or a mixture of polymers that can adhere to a wet mucous surface. These adhesives may be formulated as ointments, hydrogels (see above) thin films, and other application forms. Often, these adhesives have the biologically active agent mixed therewith to effectuate slow release or local delivery of the active agent. Some are formulated with additional ingredients to facilitate penetration of the active agent through the nasal mucosa, e.g., into the circulatory system of the individual.

Acrylic-based hydrogels are well-suited for bioadhesion due to their flexibility and nonabrasive characteristics in the partially swollen state which reduce damage-causing attrition to the tissues in contact [Park, et al., J. Control. Release 2:47-57, 1985]. Furthermore, their high permeability in the swollen state allows unreacted monomer, un-crosslinked polymer chains, and the initiator to be washed out of the matrix after polymerization, which is an important feature for selection of bioadhesive materials for use within the invention.

A particularly useful bioadhesive agent within the coordinate administration, and/or combinatorial formulation methods and compositions of the invention is chitosan, as well as its analogs and derivatives. Chitosan is a non-toxic, biocompatible and biodegradable polymer that is widely used for pharmaceutical and medical applications because of its favorable properties of low toxicity and good biocompatibility.

As further provided herein, the methods and compositions of the invention will optionally include a chitosan derivative or chemically modified form of chitosan. One such novel derivative for use within the invention is denoted as a ≈-[1→4]-2-guanidino-2-deoxy-D-glucose polymer (poly-GuD). Chitosan is the N-deacetylated product of chitin, a naturally occurring polymer that has been used extensively to prepare microspheres for oral and intra-nasal formulations. The chitosan polymer has also been proposed as a soluble carrier for parenteral drug delivery. Within one aspect of the invention, o-methylisourea is used to convert a chitosan amine to its guanidinium moiety.

Formulation and Administration

Mucosal delivery formulations of the present invention comprise the biologically active agent to be administered typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

The compositions and methods of the invention may be administered to subjects by a variety of mucosal administration modes, including by oral, rectal, vaginal, intranasal, intrapulmonary, or transdermal delivery, or by topical delivery to the eyes, ears, skin or other mucosal surfaces. Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers. In some embodiments of the present invention, the nasal spray solution further comprises a propellant. The pH of the nasal spray solution is optionally between about pH 6.8 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 4-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, mucosal formulations are administered as dry powder formulations comprising the biologically active agent in a dry, usually lyophilized, form of an appropriate particle size, or within an appropriate particle size range, for intranasal delivery. Minimum particle size appropriate for deposition within the nasal or pulmonary passages is often about 0.5μ mass median equivalent aerodynamic diameter (MMEAD), commonly about 1μ MMEAD, and more typically about 2μ MMEAD. Maximum particle size appropriate for deposition within the nasal passages is often about 10μ MMEAD, commonly about 8μ MMEAD, and more typically about 4μ MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which rely on the patient's breath, upon pulmonary or nasal inhalation, to disperse the power into an aerosolized amount. Alternatively, the dry powder may be administered via air assisted devices that use an external power source to disperse the powder into an aerosolized amount, e.g., a piston pump.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically 1/2 to 2, and most often ¾ to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10,000 and preferably not more than 3,000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g., oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the invention, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin.

The term “subject” as used herein means any mammalian patient to which the compositions of the invention may be administered.

Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more biologically active agent formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

### EXAMPLES

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Lipids Screened for Their Ability to Enhance the Permeation of Biological Agents Across an Epithelial Cell Monolayer

The present example presents a list of lipids screened for their ability to enhance the permeation of a biological agent across and epithelial cell monolayer in vitro.

Tight junction integrity of human epithelial tissue can be assayed in vitro by measuring the level of electrical resistance and degree of sample permeation. A reduction in electrical resistance and enhanced permeation suggests that the tight junctions have been compromised and openings have been created between the epithelial cells. In effect, lipids that induce a measured reduction in electrical resistance across a tissue membrane, referred to as (TER) reduction, and enhance the permeation of a small molecule through a tissue membrane (paracellular transport) are classified as TJMLs. In addition, TER, sample permeation, LDH recovery and the level of cell toxicity and/or cell viability for TJMLs are also assessed to determine whether select lipids could function as tight junction modulating lipids for the delivery of a biological agent across a mucosal surface, for example intranasal (IN) drug delivery. TER recovery measures whether the effect on epithelial junctional structure and/or physiology is reversible, which is critical in preventing damage to the mucosal cell layer and reducing the possibility of infection. Further, the above described assay can measure transcellular transport (transport through the cell) of molecules and/or biological agents across an epithelia.

The assays used to screen the exemplary lipids of the present invention are described in Example 2. Table 1 provides the common name, chemical name and the molecular weight for a subset of lipids screened in this application. Lipids marked with “*” within Table 1 were purchased from Avanti Polor Lipids, Incorporated (Alabaster, Ala.). Lipids marked with were purchased from Biomol International (Plymouth Meeting, Pa.).

TABLE 1
Lipids Screened for Permeation Enhancing Activity

| Lipid Name | Chemical Name or Other Name | Molecular Weight |
|---|---|---|
| POVPC* | 1-Palmitoyl-2-(5′-oxo-Valeroyl)-sn-Glycero- | 593.74 |
| | 3-Phosphocholine | |
| PGPC | 1-Palmitoyl-2-Glutaroyl-sn-Glycero-3- | |
| | Phosphocholine | |
| Sphingomyelin (brain | (2S,3R,4E)-2-Acylaminooctadec-4-ene-3- | |
| porcine) | Hydroxy-1-Phosphocholine | |
| Ceramide (brain | (2S,3R,4E)-2-Acylamino-1,3-Octadec-4- | |
| porcine) | Enediol | |
| Cerebroside (brain | Total Cerebrosides | |
| porcine) | | |
| Cerebroside Sulfatide | NH₄,HSO₄-3Galβ1-1′Ceramide | |
| (porcine) | | |
| Porcine brain | Total Brain Ganglioside with various | |
| ganglioside | saccharidic headgroup | |
| Platelet-Activation | 1-Alkyl-2-Acetoyl-sn-Glycero-3- | |
| Factor | Phosphocholine | |
| Lyso-PAF | 1-Alkyl-2-Hydroxy-sn-Glycero-3- | |
| | Phosphocholine | |
| Phosphatidylinositol | L-α-Phosphatidylinositol Sodium Salt | |
| (bovine) | | |
| Phosphatidylinositol | L-α-Phosphatidylinositol Sodium Salt | |
| (Soy) | | |
| Cardiolipin (sodium | 1,3-Di(3-sn-Phosphatidyl)-sn-Glycerol | |
| salt) | Disodium Salt | |
| Sphingosine-1- | (2S,3R,4E)-2-Aminooctadec-4-ene-1,3-Diol- | |
| phosphate | 1-Phosphate | |
| Dimethylsphingosine | (2S,3R,4E)-2-Dimethylaminooctadec-4-Ene- | |
| | 1,3-Diol | |
| Trimethylsphingosine | (2S,3R,4E)-2-Trimethylaminooctadec-4-Ene- | |
| | 1,3-Diol (Chloride Salt) | |
| Glucosyl-sphingosine | D-Glucosyl-β1-1′-D-erythro-Sphingosine | |
| Galactosyl | D-Galactosyl-β1-1′-D-erythro-Sphingosine | |
| sphingosine | | |
| N-acetoyl ceramide-1- | (2S,3R,4E)-2-Acetoylaminooctadec-4-Ene- | |
| phosphate | 1,3-Diol-1-Phosphate (Ammonium Salt) | |
| N-octanoyl ceramide- | (2S,3R,4E)-2-Octanoylaminooctadec-4-Ene- | |
| 1-phosphate | 1,3-Diol-1-Phosphate (Ammonium Salt) | |
| 3-beta-hydroxy- | 3β-Hydroxy-5α-Cholest-8(14)-en-15-one | |
| 5alpha-cholest-8(14)- | | |
| en-15-one | | |
| 1,2-di-O-phytanyl- | 1,2-Di-O-Phytanyl-Glycero-3-Phosphocholine | |
| glycero-3- | | |
| phosphocholine | | |
| 1,2-Dioleoyl-sn- | 1,2-Dioleoyl-sn-Glycero-3- | |
| Glycero-3- | Ethylphosphocholine | |
| Ethylphosphocholine | | |
| 16:0-09:0(COOH)PC | 1-Palmitoyl-2-Azelaoyl-sn-Glycero-3- | |
| | Phosphocholine | |
| 16:0-09:0(ALDO)PC | 1-Palmitoyl-2-(9′-oxo-Nonanoyl)-sn-Glycero- | |
| | 3-Phosphocholine | |
| Lactosyl(β) | D-Lactosyl-β1-1′-D-erythro-Sphingosine | |
| Sphingosine | | |
| Azelaoyl PAF (C16- | 1-O-Hexadecyl-2-Azelaoyl-sn-Glycero-3- | 651.86 |
| 09:0)* | Phosphocholine | |
| C16 Lyso-PAF* | 1-O-Hexadecyl-2-Hydroxy-sn-Glycero-3- | 481.65 |
| | Phosphocholine | |
| C18 Lyso-PAF* | 1-O-Octadecyl-2-Hydroxy-sn-Glycero-3- | 509.71 |
| | Phosphocholine | |
| C18-02:0 PC(C18 | 1-O-Octadecyl-2-Acetoyl-sn-Glycero-3- | 551.74 |
| PAF)* | Phosphocholine | |
| C16-04:1 PC* | 1-O-Hexadecyl-2-Butenoyl-sn-Glycero-3- | 549.73 |
| | Phosphocholine | |
| C16-04:0 PC* | 1-O-Hexadecyl-2-Butyroyl-sn-Glycero-3- | 551.74 |
| | Phosphocholine | |
| C16 Enantiomeric | 3-O-Hexadecyl-2-Acetoyl-sn-Glycero-1- | 523.69 |
| PAF* | Phosphocholine | |
| 16:0-02:0 PC* | 1-Palmitoyl-2-Acetoyl-sn-Glycero-3- | 537.67 |
| | Phosphocholine | |
| C16-02:0 PC(C16 | 1-O-Hexadecyl-2-Acetoyl-sn-Glycero-3- | 523.69 |
| PAF)* | Phosphocholine | |
| 18:0-1:0 Diether PC* | 1-O-Octadecyl-2-O-Methyl-sn-Glycero-3- | 523.73 |
| | Phosphocholine | |
| C16-22:6 PC* | 1-O-Hexadecyl-2-Docosahexaenoyl-sn- | 792.13 |
| | Glycero-3-Phosphocholine | |
| C16-20:4 PC* | 1-O-Hexadecyl-2-Arachidonoyl-sn-Glycero- | 768.11 |
| | 3-Phosphocholine | |
| C16-20:5 PC* | 1-O-Hexadecyl-2-Eicosapentaenoyl-sn- | 766.1 |
| | Glycero-3-Phosphocholine | |
| C16-02:0 DG* | 1-O-Hexadecyl-2-Acetoyl-sn-Glycerol | 358.56 |
| C16-18:1 PC* | 1-O-Hexadecyl-2-Oleoyl-sn-Glycero-3- | 746.1 |
| | Phosphocholine | |
| C18-04:0 PC* | 1-O-Octadecyl-2-Butyroyl-sn-Glycero-3- | 579.8 |
| | Phosphocholine | |
| 2-O-Ethyl-PAF⁺ | 1-O-Hexadecyl-2-O-Ethyl-sn-Glycero-3- | 509.7 |
| | Phosphorylcholine | |
| C-PAF⁺ | 1-O-Hexadecyl-2-N-Methylcarbamyl-sn- | 538.7 |
| | Glycero-3-Phosphocholine | |
| PAF-antangonist⁺ | 1-O-Hexadecyl-2-O-Acetyl-sn-Glycero-3- | 579.8 |
| | Phospho(N,N,N-trimethyl) Hexanolamine | |
| 2-O-Methyl-PAF⁺ | 1-O-Hexadecyl-2-O-Methyl-sn-Glycero-3- | 495.7 |
| | Phosphorylcholine | |

The lipids presented above in Table 1 were dissolved in phosphate buffered saline (PBS) directly, or in chloroform followed by evaporation in a laminar flow hood and then re-suspended in PBS, Buffer I or Buffer II, or dissolved in 95% ethanol, or dissolved in 20% ethanol. Alternatively, sonication or a pneumatic actuator (LipoFast™, supplied by Avestin Inc.) was used to facilitate dissolution of the lipid into liposome form. Briefly, the LipoFast™ procedure produces unilamellar liposome by the manual extrusion of multilamellar liposome suspension through a polycarbonate membrane of define pore size, using gas-tight-glass syringes. To accomplish this, the sample is passed back and forth through the membrane several times by force applied by two syringes that flank the chamber containing the membrane. A clear solution as seen within the glass syringes indicates that the micelle size is less than 100 nM. Micelle sizes that exceed 100 nM will appear milky.

### Example 2

### In Vitro Methods Employed to Assess the Ability of Lipids to Enhance the Permeation of a Biological Agent Across an Epithelial Cell Monolayer

The present example illustrates the methods and procedures used to assess the efficacy of each lipid in Table 1 to enhance the permeation of a biological agent across an epithelial cell monolayer. The lipids were assayed for their effect on transepithelial electrical resistance (TER), TER recovery, lactate dehydrogenase (LDH) levels or cytotoxicity, sample permeation. LDH recovery was also assessed for certain lipids. The results from the individual assays were obtained after treatment with a a single lipid followed by collection of the basolateral medium to measure sample permeation, collection of the apical treatment media to measure LDH release to characterize cytotoxicity and TER measurements to assess changes in electrical resistance. The cell culture conditions and protocols for each assay are explained below in detail. Although the protocols are described in detail, they may be modified accordingly. Also described are the reagents used in the subsequent Examples.

Cell Cultures

Normal, human-derived tracheal/bronchial epithelial cells will serve as the model cell system for assessing the lipids listed in Table 1. The cells are supplied by MatTek Corp. (Ashland, Mass.) as the EpiAirway™ Tissue Model. The cells are provided as a confluent monolayer on a Millipore Milicell-CM cell culture insert with a pore size of 0.4 μM, inner diameter of 0.8 cm and surface area of 0.6 cm² and comprised of transparent hydrophilic Teflon (PTFE). Upon receipt, the membranes are cultured in 1 ml basal media (phenol red-free and hydrocortisone-free Dulbecco's Modified Eagle's Media (DMEM) at 37° C./5% CO₂ for 24-48 hours before use. Inserts are feed for each day of recovery.

Measurement of Transepithelial Electrical Resistance (TER)

TER measurements were accomplished using the Endohm-12 Tissue Resistance Measurement Chamber connected to the EVOM Epithelial Voltohmmeter (World Precision Instruments, Sarasota, Fla.) with the electrode leads. The electrodes and a tissue culture blank insert were equilibrated for at least 20 minutes in MatTek medium with the power off prior to checking calibration. The background resistance was measured with 1.5 ml media in the Endohm tissue chamber and 300 μl media in the blank insert. The top electrode was adjusted so that it was close to, but not making contact with, the top surface of the insert membrane. Background resistance of the blank insert was about 5-20 ohms. For each TER determination, 300 μl of MatTek medium was added to the insert followed by placement in the Endohm chamber. TER values are a function of the surface area of the tissue. An example of how TER was calculated is as follows:

$$Nominal Resistance , Ohm * {cm}^{2} = ( TERt - blank ) * 0.12$$

$$Relative TER , \% = \frac{TERt - blank}{TER 0 - blank} \times 100$$

Where transepithelial electrical resistance at time t=TERₜ and blank refers to the TER of an empty insert. By this method of calculation, TER will be expressed as both Ohms*cm2 and percent original TER value.

TER recovery was calculated as described in the above paragraph.

Cell Viability (MTT Assay)

Cell viability will be assessed using the MTT assay (MTT-100, MatTek kit). This kit measures the uptake and transformation of tetrazolium salt to formazan dye. Thawed and diluted MTT concentrate is prepared 1 hour prior to the end of the dosing period with the lipid by mixing 2 mL of MTT concentrate with 8 mL of MTT diluent. Each cell culture insert is washed twice with PBS containing Ca⁺² and Mg⁺² and then transferred to a new 96-well transport plate containing 100 μL of the mixed MTT solution per well. This 96-well transport plate is then incubated for three hours at 37° C. and 5% CO₂. After the three hour incubation, the MTT solution is removed and the cultures are transferred to a second 96-well feeder tray containing 250 μL MTT extractant solution per well. An additional 150 μL of MTT extractan solution was added to the surface of each culture well and the samples sat at room temperature in the dark for a minimum of two hours and maximum of 24 hours. The insert membrane was then pierced with a pipet tip and the solutions in the upper and lower wells were allowed to mix. Two hundred microliters of the mixed extracted solution along with extracted blanks (negative control) was transferred to a 96-well plate for measurement with a microplate reader. The optical density (OD) of the samples was measured at 570 nm with the background subtraction at 650 nm on a plate reader. Cell viability was expressed as a percentage and calculated by dividing the OD readings for treated inserts by the OD readings for the PBS treated inserts and multiplying by 100. For the purposes of this assay, it was assumed that PBS had no effect on cell viability and therefore represented 100% cell viability.

Cytotoxicity (LDH Assay)

The amount of cell death was assayed by measuring the loss of lactate dehydrogenase (LDH) from the cells using a CytoTox 96 Cytotoxicity Assay Kit (Promega Corp., Madison, Wis.). A treatment of 1% Octylphenolpoly(ethyleneglycolether)x (Triton X-100™) diluted in PBS was used as a lysis control. One percent Triton X-100™ mediated cell lysis was normalized to 100%. For basal-lateral LDH levels, triplicates of 50 μl of the basal media were loaded into a 96-well assay plate. For apical LDH levels, 150 μl of Epi-Cm was added to the apical side of each chamber and mixed by pipeting. One hundred and fifty microliters was then removed and diluted 2-fold prior to performing the LDH assay. All apical LDH assay were performed in triplicate and with 50 μl of the diluted test solution. Fresh, cell-free culture medium will be used as a blank. Total LDH levels were determined by lysing cells in a final concentration of 0.9% Triton-X100™. Fifty microliters of substrate solution was added to each well and the plates incubated for 30 minutes at room temperature in the dark. Following incubation, 50 μl of stop solution was added to each well and the plates read on an optical density plate reader at 490 nM. Cytotoxicity was expressed as a percentage calculated by subtracting the average absorbance of the PBS control wells as the endogenously released LDH level and expressing that value relative to the average Triton-X100 control, which represents total LDH content.

$$Relative Cytotoxicity , \% = \frac{ODx - ODpbs}{ODtriton} \times 100$$

Osmolality

Samples will be measure by Model 20200 from Advanced Instruments Inc. (Norwood, Mass.).

FITC (fluorescein-5-isothiocyanate)-Dextran Permeation Assay

Each tissue insert was placed in an individual well containing 1 ml of MatTek basal media. On the apical surface of the inserts, 20 μl of test formulation was applied according to study design, and the samples were placed on a shaker (˜100 rpm) for 1.5 hours at 37° C. FITC-labeled dextran solution was added to inserts apically and a fluorescence measurement was taken from the basolateral media after the incubation period. Two hundred microliters of the basal media for each test formulation was transferred to a dark-wall fluorescent reading plate. Each test formulation was tested in triplicate. Fluorescent intensity was measured at 470 nM with the microplate fluorescence reader FLx800 (Bio-Tek Instruments, Inc., Winooski, Vt.). A FITC labeled dextran with a molecular weight of 3 kDA, 10 kDA, 20 kDA, 40 kDA, 70 kDA and/or 500 kDA was used to assess the ability of individual lipids to deliver a model protein across an epithelia.

Permeation is expressed as percent permeation and was calculated as follows:

$$\% Permeation = \frac{Cb \times Vb}{Ca \times Va} \times 100$$

$$Apparent Permeability ( Papp ) , cm \text{/} sec = \frac{Vb}{SA \times Ca} \frac{Cb}{dt}$$

Terms

Basolateral PYY Concentration: Cb

Apical PYY Concentration: Ca

Basolateral Volume: Vb

Apical Volume: Va

Filter Surface Area: SA

Elapsed Time: dt

Reagents

Table 2 illustrates the sample reagents used in the subsequent Examples of the present application.

TABLE 2
Sample Reagents

| Reagent | Grade | Manufacturer | City, State | Lot # | MW |
|---|---|---|---|---|---|
| 1XDPBS++ | TC | Gibco/Invitrogen ™ | Carlsbad, CA | 1213061 | |
| Sterile, Nulcease-Free Water | | Ambion ™ | Austin, TX | 065P053618A | |
| Air-100 Medium | TC | MatTek ™ | Ashland, MA | 11110565 | |
| Air-196 inserts | | MatTek ™ | Ashland, MA | 7118 | |
| CytoTox 96 Assay | | Promega ™ | Madison, WI | 210634 | |
| Chloroform | | Sigma ™ | St. Louis, MO | 094K3725 | |
| Cholorbutanol, anhydrous | NF | Spectrum ™ | New | RI1646 | |
| | | | Brunswick, NJ | | |
| Methyl-b-Cyclodextrin | | Sigma ™ | St.Louis, MO | 023K1202 | |
| L-a-Phospharidycholine | | Sigma ™ | St.Louis, MO | 55H8377 | |
| Didecanoyl | | | | | |
| Edetate Disodium | USP | Dow Chemicals ™ | | 1034N-00269-2 | |
| Sodium Citrate, Dihydrate | USP | Spectrum ™ | New | RH1056 | |
| | | | Brunswick, NJ | | |
| Citric Acid, Anhydrous | USP | Sigma ™ | St.Louis, MO | 062K003 | |
| a-Lactose monohydrate | NF | Spectrum ™ | New | RJ1103 | |
| | | | Brunswick, NJ | | |
| Sorbitol | NF | Spectrum ™ | New | QE0194 | |
| | | | Brunswick, NJ | | |
| PYY 3-36 | GMP | Bachem ™ | Torrance, CA | FYY3360301A | |
| Human Insulin, Recombinant, | USP | Diosynth ™ | Sioux City, IA | SIHR902 | |
| GMP | | | | | |
| 2N Hydrochloric Acid | Research | JT Baker ™ | Philpsburg, NJ | B18512 | |
| 2N Sodium Hydroxide | Research | JT Baker ™ | Philpsburg, NJ | B06503 | |
| FITC-Dextran 3,000 | Research | Molecular Probes ™ | Carlsbad, CA | 41675A | |
| FITC-Dextran 10,000 | Research | Molecular Probes ™ | Carlsbad, CA | 37974A | |
| FITC-Dextran 40,000 | Research | Molecular Probes ™ | Carlsbad, CA | 37974A | |
| FITC-Dextran 70,000 | Research | Molecular Probes ™ | Carlsbad, CA | | |
| FITC-Dextran 500,000 | Research | Molecular Probes ™ | Carlsbad, CA | 36410A | |

### Example 3

### Lipid Permeation Kinetics

The present example demonstrates that examplary lipids of the present invention enhance epithelia permeation. Several different lipid types (see Table 1) were screened to select for lipids that are capable of enhancing the permeation of a biological agent across an epithelial cell monolayer. To select for permeation enhancing lipids, each lipid was tested for its ability to reduce electrical resistance of a monolayer of human-derived tracheal/bronchial epithelial cells (EpiAirway™ Model System) assayed by TER (refer to Example 2 for protocol details). A reduction in TER correlates with the ability to enhance the permeation of a molecule and biological agent across an epithelia. Tables 3 and 4 represent the initial screen of the lipids listed in Table 1. These tables show the measured TER reduction and cytotoxicity (Cytotoxic Effect) data for the lipids listed in Table 1. Further, Table 4 shows the permeation of FITC-dextran 3000 (FD3) across an epithelia.

For the instant application, phosphate buffered saline (PBS) served as a negative control for both the TER assay and LDH (cytotoxicity) assay. PN159 is here used at 25 μM concentration as a positive control effective at reducing TER. PN159 refers to a formulation containing a permeability enhancer previously found to be effective in reducing TER but not inducing significant cell cytotoxicity. Special Sauce was also used as a positive control effective at reducing TER but not inducing significant cell cytotoxicity. Special Sauce used herein consists of 45 mg/mL methyl-β-cyclodextrin, 1 mg/mL 1,2-Dimyristoylamido-1,2-deoxyphosphatidylcholine (DDPC) and 1 mg/mL ethylene diamine tetraacetic acid (EDTA). Additionally, 0.3% or 1% Triton-X100 served as a positive control for both TER measurements and cytotoxicity (LDH) because it is effective at reducing TER and increasing LDH levels in the cell media. TER measurements and LDH levels were taken immediately after a one hour treatment of the cultured cells with each lipid, unless specified otherwrise.

TER reduction was expressed as the percent decrease in TER value from time zero to one hour post-treatment. Thus, greater percent reduction in TER represents less electrical resistance across the epithelial cell monolayer and consequently greater epithelial cell permeation. Cytotoxicity (LDH levels) for each lipid was expressed as a percent of the LDH levels measured after Triton-X100 treatment of the cells. Triton-X100™ LDH levels were normalized to 100%.

TABLE 3
Percent TER and LDH of an Epithelia in the Presence of Lipids

| | | | | Mean TER | Cytotoxic Effect |
|---|---|---|---|---|---|
| | | | | Reduction 1 hr. | (LDH) |
| | | | | Post- | 1 hr. Post- |
| | Lipid Name or Control | Concentration | | treatment | treatments |
| Negative Controls | Hypotonic PBS | N/A | | 22% | 2% |
| | Isotonic PBS | N/A | | 18% | 2% |
| | 2% Ethanol | N/A | | 25% | 2% |
| Positive Controls | PN159 | 25 | μM | 87% | 17% |
| | Special Sauce | N/A | | 91% | 16% |
| | 0.3% Triton-X100 | N/A | | 100% | 100% |
| LIPIDS | POVPC | 1000 | μM | 93% | 21% |
| | | 500 | μM | 87% | 11% |
| | | 250 | μM | 52% | 5% |
| | | 125 | μM | 32% | 2% |
| | | 62.5 | μM | 23% | 2% |
| | PGPC | 1000 | μM | 92% | 21% |
| | | 500 | μM | 80% | 13% |
| | | 250 | μM | 48% | 4% |
| | | 125 | μM | 28% | 2% |
| | | 62.5 | μM | 21% | 2% |
| | Azelaoyl PAF | 1000 | μM | 95% | 26% |
| | (C16-09:0) | 500 | μM | 93% | 16% |
| | | 250 | μM | 93% | 10% |
| | | 125 | μM | 76% | 5% |
| | | 62.5 | μM | 41% | 2% |
| | Lyso-Platelet-Activation | 1000 | μM | 84% | 34% |
| | Factor | 500 | μM | 61% | 22% |
| | | 250 | μM | 29% | 6% |
| | | 125 | μM | 23% | 3% |
| | | 62.5 | μM | 48% | 2% |
| | Platelet-Activation Factor | 1000 | μM | 35% | 19% |
| | Galactosyl sphingosine | 1000 | μM | 91% | 26% |
| | | 500 | μM | 42% | 2% |
| | | 250 | μM | 43% | 3% |
| | | 125 | μM | 36% | 2% |
| | N-acetoyl ceramide-1- | 1000 | μM | 42% | 2% |
| | phosphate | 500 | μM | 29% | 2% |
| | | 250 | μM | 23% | 2% |
| | | 125 | μM | 24% | 2% |
| | Sphingomyelin (brain | 1000 | μM | 31% | 2% |
| | porcine) | | | | |
| | Lactosyl(β) Sphingosine | 1000 | μM | 95% | 14% |
| | Cardiolipin (sodium salt) | 1000 | μM | 32% | 21% |
| | 16:0-09:0(COOH) | 500 | μM | 92% | 9% |
| | Phosphocholine | | | | |
| | 16:0-09:0(ALDO) | 1000 | μM | 81% | 10% |
| | Phosphocholine | | | | |
| | N-acetoyl ceramide-1- | 1000 | μM | 42% | 2% |
| | phosphate | 500 | μM | 29% | 2% |
| | | 250 | μM | 23% | 2% |
| | | 125 | μM | 24% | 2% |
| | 18:0-1:0 Diether PC | 1000 | μM | 99% | 50% |
| | | 500 | μM | 89% | 26% |

For the data in Table 3, the negative controls had no significant effect on TER (18% to 25% TER reduction) while the positive control PN159 reduced TER by 85%. Also, shown is the 0.3% Triton-X100 positive control which reduced TER by 100%. Furthermore, the positive controls including 25 μM PN159 and Special Sauce did not induce a cytotoxic effect (i.e., the LDH levels for the controls remained less than 30% of the Triton-X100 LDH levels).

A majority of the lipids listed in Table 3 failed to reduce TER beyond that of the negative controls. Furthermore, several lipids reduced TER significantly but induced a cytotoxic effect.

POVPC was also assayed for its effect on cell viability (MTT assay). The data (not shown) shows that POVPC did not reduce cell viability below that of the control Special Sauce.

The lipids 1-Palmitoyl-2-(5′-oxo-Valeroyl)-sn-Glycero-3-Phosphocholine (POVPC); 1-Palmitoyl-2-Glutaroyl-sn-Glycero-3-Phosphocholine (PGPC); 1-O-Hexadecyl-2-Azelaoyl-sn-Glycero-3-Phosphocholine (Azelaoyl PAF ((C16-09:0)); 1-Alkyl-2-Hydroxy-sn-Glycero-3-Phosphocholine (Lyso-PAF); Galactosyl sphingosine; Lactosyl(P) sphingosine; 16:0-09:0(COOH) phosphocholine and 16:0-09:0(ALDO) phosphocholine reduced TER by 80% or more and maintained LDH levels below about 30% suggesting that these lipids may function as permeation enhancers without causing any significant cytotoxic effects.

For the data in Table 4, TER reduction is expressed as the percent of the original TER value at time zero, thus a lower percent TER value equates to a greater TER reduction.

TABLE 4
Percent TER, LDH and FD3 Permeation of an Epithelia in the Presence of Lipids

| | | | | Mean % | |
|---|---|---|---|---|---|
| | | | Mean | Relative | Mean |
| | Lipid Name or | | % of Original | Cytotoxic Effect | % FD3 |
| | Control | Concentration | TER Value | (LDH) | Permeation |
| Negative | PBS/Chloroform | 0.75X | 93% | 0% | 0% |
| Controls | PBS | 0.75X | 93% | −1% | 0% |
| Positive | Special Sauce | 1X | −6% | 36% | 24% |
| Controls | 1% TritonX- | ND | −7% | 100% | ND |
| | 10 ™ | | | | |
| LIPIDS | Azelaoyl PAF | 1000 μM | −5% | 3% | 9% |
| | (C16-09:0) | | | | |
| | C16 Lyso-PAF | 1000 μM | 14% | 26% | 6% |
| | (POVPC) | 1000 μM | 0% | 9% | 10% |
| | C18 Lyso-PAF | 1000 μM | 40% | 17% | 2% |
| | C18-02:0 | 1000 μM | 1% | 20% | 8% |
| | PC(C18 PAF) | 500 μM | 44% | 8% | 2% |
| | C16-04:1 PC | 1000 μM | −2% | 27% | 12% |
| | | 500 μM | 11% | 18% | 6% |
| | C16-04:0 PC | 1000 μM | 0% | 22% | 7% |
| | C16 | 1000 μM | 1% | 35% | 11% |
| | Enantiomeric | | | | |
| | PAF | | | | |
| | 16:0-02:0 PC | 1000 μM | 25% | 23% | 8% |
| | C16-02:0 | 1000 μM | 2% | 32% | 14% |
| | PC(C16 PAF) | 500 μM | 27% | 20% | 5% |
| | 18:0-1:0 Diether | 1000 μM | 111% | −1% | 0% |
| | PC | | | | |
| | C16-22:6 PC | 1000 μM | 97% | −1% | 0% |
| | C16-20:4 PC | 1000 μM | 96% | 0% | 0% |
| | C16-20:5 PC | 1000 μM | 90% | −1% | 0% |
| | C16-02:0 DG | 1000 μM | 98% | −3% | 0% |
| | C16-18:1 PC | 1000 μM | 86% | −2% | 0% |
| | C18-04:0 PC | 1000 μM | 85% | −3% | 0% |
| | PAF-antagonist | 1000 μM | 9% | 10% | 11% |
| | | 500 μM | 20% | 8% | 6% |
| | 2-O-Methyl-PAF | 1000 μM | 2% | 20% | 16% |
| | | 500 μM | 8% | 18% | 7% |
| | 2-O-Ethyl-PAF | 1000 μM | 70% | 2% | 2% |
| | | 500 μM | 112% | 1% | 1% |
| | C-PAF | 1000 μM | 2% | 15% | 12% |
| | | 500 μM | 10% | 11% | 11% |

For the data in Table 4, the following lipids enhanced the permeation of FD3 above that of the negative controls through an epithelial cell monolayer: 1-O-Hexadecyl-2-Azelaoyl-sn-Glycero-3-Phosphocholine (Azelaoyl PAF (C16-09:0)); 1-O-Hexadecyl-2-Hydroxy-sn-Glycero-3-Phosphocholine (C16 Lyso-PAF); 1-Palmitoyl-2-(5′-oxo-Valeroyl)-sn-Glycero-3-Phosphocholine (POVPC); 1-O-Octadecyl-2-Hydroxy-sn-Glycero-3-Phosphocholine (C18 Lyso-PAF); 1-O-Octadecyl-2-Acetoyl-sn-Glycero-3-Phosphocholine (C18-02:0 PC (C18 PAF)); 1-O-Hexadecyl-2-Butenoyl-sn-Glycero-3-Phosphocholine (C16-04:1 PC); 1-O-Hexadecyl-2-Butyroyl-sn-Glycero-3-Phosphocholine (C16-04:0 PC); 3-O-Hexadecyl-2-Acetoyl-sn-Glycero-1-Phosphocholine (C16 Enanteomeric PAF); 1-O-Hexadecyl-2-Acetoyl-sn-Glycero-3-Phosphocholine (C16-02:0 PC (C16 PAF)); 1-O-hexadecyl-2-O-Acetyl-sn-Glycero-3-Phospho(N,N,N-trimethyl) Hexanolamine (PAF-antagonist); 1-O-Hexadecyl-2-O-Methyl-sn-Glycero-3-Phosphorylcholine (2-O-Methyl-PAF); 1-O-Hexadecyl-2-O-Ethyl-sn-Glycero-3-Phosphorylcholine (2-O-Ethyl-PAF) and 1-O-Hexadecyl-2-N-Methylcarbamyl-sn-Glycero-3-Phosphocholine (C-PAF). Several of these lipids were further tested to determine dose-dependent effects (see below).

The data in Table 4 show that a subset of the lipids screened enhance the permeation of the FD3 molecule across and epithelial cell monolayer indicating that not all the lipids tested promote the permeation of small molecules across an epithelial cell monolayer. The lipids C18 PAF, C16 PAF and C16:04-1PC were assayed for their effect on cell viability (MTT assay). The data (not shown) indicates that all three lipids did not reduce cell viability below that of Special Sauce (control).

The lipids 1-O-Hexadecyl-2-Azelaoyl-sn-Glycero-3-Phosphocholine (Azelaoyl PAF (C16-09:0)); 1-O-Hexadecyl-2-Hydroxy-sn-Glycero-3-Phosphocholine (C16 Lyso-PAF); 1-Palmitoyl-2-(5′-oxo-Valeroyl)-sn-Glycero-3-Phosphocholine (POVPC); 1-O-Octadecyl-2-Hydroxy-sn-Glycero-3-Phosphocholine (C18 Lyso-PAF); 1-O-Octadecyl-2-Acetoyl-sn-Glycero-3-Phosphocholine (C18-02:0 PC (C18 PAF)); 1-O-Hexadecyl-2-Butenoyl-sn-Glycero-3-Phosphocholine (C16-04:1 PC); 1-O-Hexadecyl-2-Butyroyl-sn-Glycero-3-Phosphocholine (C16-04:0 PC); 3-O-Hexadecyl-2-Acetoyl-sn-Glycero-1-Phosphocholine (C16 Enanteomeric PAF) and 1-O-Hexadecyl-2-Acetoyl-sn-Glycero-3-Phosphocholine (C16-02:0 PC (C16 PAF)) were further tested within a concentration range of 250 μM to 1000 μM. Cytotoxicity (LDH levels) for each lipid was expressed as a percent of the LDH levels measured after TritonX-100™ treatment of the cells. TritonX-100™ LDH levels were normalized to 100%. A greater mean percent of LDH indicates a higher level of cytotoxicity while a lesser mean percent TER indicates a greater TER reduction.

As expected, the negative control PBS had no significant effect on TER (77% of original TER value) while the positive controls PN159 and Special Sauce decreased TER to 8% and −3% of the original TER value (i.e., pre-treatment), respectively. Also, the 1% TritonX-100™ positive control reduced TER (−6%). Furthermore, PBS exhibited no relative cytotoxic effect (0%). Special Sauce and PN159 did not induce a significant cytotoxic effect (i.e., the LDH levels for the controls remained less than about 30% of the TritonX-100™ LDH levels).

A dose-dependent effect was observed with the higher lipid concentrations inducing a greater reduction in TER. Furthermore, all but one lipid (C16-04: 1 PC at 1000 μM) reduced TER with minimal effect on LDH levels indicating the lipids compromise epithelial tight junction integrity without causing a significant cytotoxic effect and, thus, show great potential has epithelial cell permeation enhancers.

Thus, these data (Table 3 and 4) show the surprising and unexpected discovery that select lipids, primarily those belongs to the the class of lipids known as PAF analogs, exhibit TER reducing and permeation enhancing properties without increasing cell cytotoxicity beyond acceptable levels of an epithelial cell monolayer. Based on these data, select lipids (“permeation enhancing lipids”) were chosen for further characterization

### Example 4

### Epithelia Recoverv Time Course

The present example demonstrates the rate at which permeation enhancing lipids reduced TER and the rate of TER recovery post-treatment. Reversibility is a critical factor in selecting epithelial cell permeabiling enhancers since the barrier function of the epithelial cells serves as the first line of defense against pathogens and the entrance of toxins into the body. The permeation enhancing lipids C16 PAF; C18 PAF; C16 Enantiomeric PAF; POVPC; C16-04: 1 PC and PGPC were incubated with a monolayer of human-derived tracheal/bronchial epithelial cells (EpiAirway™ Model System) and TER measurements taken either immediately following the incubation time or 20 to 24 hours post-treatment. The lipid glucosyl sphingosine was also tested. Each permeation enhancing lipid (except PGPC) was applied at a concentration of 1000 μM for 15, 30 and 60 minutes. The permeation enhancing lipid PGPC and the lipid glucosyl sphingosine were applied at a concentration of 500 μM for 1, 3, 5, 30 and 60 minutes. TER measurements were taken immediately after each application to determine how quickly each lipid could reduce TER.

Lipids C16 PAF, C18 PAF, C16 Enantiomeric PAF, C16-04-PC and POVPC were assayed for their effect on TER after a 15 minutes, 30 minute and 60 minute incubation with the epithelial airway model system (EpiAirway™). The data indicates that within 15 minutes C16 PAF, C18 PAF, C16 Enantiomeric PAF and C16-04-PC reduced TER to levels equivalent to that of the Triton-X100™ control suggesting that these lipids are fast acting in their ability to promote permeation of an epithelia. The TER reduction observed at 30 minutes and 60 minutes was equivalent to the 15 minute TER reduction for C16 PAF, C18 PAF, C16 Enantiomeric PAF and C16-04-PC. Further, a time-dependent permeation of FD3 was observed with C16 PAF, C18 PAF, C16 Enantiomeric PAF, C16-04-PC whereby the observed permeation was about 2% to 6% for these lipids at 15 minutes and climbed to about 10% to 36% by 60 minutes. LDH levels remained below 30% for each incubation time period tested for C16 PAF, C18 PAF, C16 Enantiomeric PAF, C16-04-PC indicating that these lipids did not induce a cytotoxic effect.

For POVPC, within 15 minutes TER was reduced to 20% below that of the PBS control and within 30 minutes TER was reduced to about 25% of the PBS control. Finally, by 60 minutes TER was reduced to levels nearly equivalent to that of the Triton-X100™ control. These data indicate that POVPC is slower acting than other C16 PAF, C18 PAF, C16 Enantiomeric and C16-04:1 PC lipids, but still maintains the ability to promote permeation of an epithelia. A time-dependent permeation of FITC-dextran 3000 (FD3) with POVPC was observed. LDH levels remained below 10% for each incubation time period tested.

For the permeation enhancing lipids C16 PAF; C18 PAF; C16 Enantiomeric PAF; POVPC and C16-04:1 PC, TER measurements were taken 20 and 24 hours post-treatment. Epithelial cells were incubated with each permeation enhancing lipid for 15, 30 or 60 minutes and TER measurements were taken at zero hour and 20 and 24 hours post-treatment. PBS served as a negative control and Triton-X100™ served as a positive control. The data indicates that all permeation enhancing lipids tested recovered within 20 hours post-treatment regardless of how long the lipid was incubated with the cells. Further, the permeation enhancing lipid POVPC showed signs of recovery within the zero hour measurement indicating that though the epithelial cells are compromised by POVPC (see TER and permeation data above in Example 3), the cells recovery quickly.

To asses how quickly the cells recovered after application and removal of the permeation enhancing lipid PGPC and the lipid glucosyl sphingosine, TER measurements were taken at 1, 3, 5, 7, and 9 hours post-treamtnet for each of the prior mentioned timed treatments (i.e., 1, 3, 5, 15, 30 and 60 minutes). TER recovery measures the reversibility of the lipid mediated effect on an epithelia. PN159 is here used at 25 μM concentration as a positive control effective at reducing TER and a TER reducing rate compartor. PN159 refers to a formulation containing a permeability enhancer previously found to be effective in reducing TER. Hyptonic PBS served as a negative control for TER reduction and TER recovery.

The TER timecourse showed that both PGPC and glucosyl sphingosine reduced TER within 1 minute while the positive control PN159 did not achieve TER reduction until 10 minutes. As expected, the PBS negative control has not significant effect on TER reduction.

The TER recovery profiles showed that the 1, 3, 5, 15 and 30 minute treatments for both PGPC and glucosyl sphingosine had comparable TER measurements within zero hour to that of the PBS negative control indicating the treated cells fully recovered within one hour. The PN159 positive control for the same treatment times did not reach PBS TER control levels until 2 hours post-treatment indicating that PN159 treated cells take twice as long compared to the lipid treated cells to fully recover. The 60 minute treatment for both lipids did not reach PBS TER control levels until three hours post-treatment indicating a delayed recover compared to the shorter length treatments. Finally, the positive conrol PN159 did not fully recover from the 60 minute treatment until 9 hours post-treatment.

These data show the surprising and unexpected discovery that the exemplary permeation enhancing lipids of the present invention compromise the integrity of an epithelial cell monolayer quickly and that this effect is reversible.

### Example 5

### Permeation Enhancing Lipids Enhance Epithelial Cell Monolayer Permeation without Adversely Effecting Cell Viability

The present example demonstrates the efficacy of the exemplary permeation enhancing lipids of the present invention to enhance the permeation of the FITC-labeled dextran molecule (FD) with a molecular weight range of 3 kD to 500 kD across a monolayer of human-derived tracheal/bronchial epithelial cells (EpiAirway™ Model System). Also, demonstrated is the effect of these permeation enhancing lipids on cell viability as assayed by MTT (refer to Example 2 for protocol details).

The data for FD permeation is summarized in Table 5. PBS and 0.3% Triton-X100™ served as negative controls. PN159 at 25 μM and “Special Sauce” served as positive control as they are both effective at enhancing the permeation of macromolecules across an epithelial cell monolayer. “Special Sauce” used herein consists of 45 mg/mL methyl-o-cyclodextrin, 1 mg/mL 1,2-Dimyristoylamido-1,2-deoxyphosphatidylcholine (DDPC) and 1 mg/mL ethylene diamine tetraacetic acid (EDTA). FD permeation was presented as the percent of FD that crossed from the apical side of the epithelial cell monolayer to the basolateral cell surface.

TABLE 5
Permeation Enhancing Lipid Mediated Permeation of FITC-Dextran

| | Lipid Name or | | % FITC-Dextran Permeation | | | | |
|---|---|---|---|---|---|---|---|
| | Control | Concentration | FD3 | FD10 | FD40 | FD70 | FD500 |
| Negative | PBS | N/A | 0% | 0.2% | 0% | 0% | 0% |
| Control | | | | | | | |
| Positive | PN159 | 25 μM | 7% | 4% | 2% | ND | ND |
| Controls | Special Sauce | N/A | 16% | 4% | 2% | ND | ND |
| Lipids | POVPC | 500 μM | 2% | ND | ND | ND | ND |
| | | 1000 μM | 10% | 3% | 1% | 0.3% | 0% |
| | PGPC | 500 μM | 10% | ND | ND | ND | ND |
| | Azelaoyl PAF | 250 μM | 1% | ND | ND | ND | ND |
| | (C16-09:0) | | | | | | |
| | Glucosyl- | 500 μM | 4% | ND | ND | ND | ND |
| | sphingosine | | | | | | |
| | 1-O-Octadecyl- | 500 μM | 5% | ND | ND | ND | ND |
| | 2-O-Methyl-sn- | | | | | | |
| | glycero-3- | | | | | | |
| | Phosphocholine | | | | | | |
| | 16:0- | 500 μM | 3% | ND | ND | ND | ND |
| | 09:0(COOH)PC | | | | | | |
| | 16:0- | 1000 μM | 0% | ND | ND | ND | ND |
| | 09:0(ALDO)PC | | | | | | |
| | Lactosyl(β) | 1000 μM | 8% | 5% | 2% | ND | ND |
| | Sphingosine | | | | | | |
| | C16-02:0 PC | 1000 μM | 22% | 6% | 2% | 2% | 0% |
| | (C16 PAF) | | | | | | |
| | C18-02:0 PC | 1000 μM | 24% | 8% | 3% | 2% | 0% |
| | (C18 PAF) | | | | | | |
| | C16-04:1 PC | 1000 μM | 25% | 8% | 3% | 0% | 0% |
| | C16 | 1000 μM | 20% | 4% | 2% | 1% | 0% |
| | Enantiomeric | | | | | | |
| | PAF | | | | | | |
| | C16 PAF | 1000 μM | 11% | 5% | 2% | ND | ND |
| | antagonist | | | | | | |
| | C16 Lyso-PAF | 1000 μM | 8% | 6% | 2% | ND | ND |

ND = no data

The negative control PBS had no effect on FD permeation (0%) while the positive controls PN159 and Special Sauce enhanced FD3 permeation 7% and 16%, respectively but had a reduced ability to enhance permeation of the larger molecular weigth FD molecules. As shown in Table 5, permeation efficacy was inversely proportional to the molecular weight of the FD molecule. The overall trend is that permeation enhancing lipids enhance the permeation of FD molecules with molecular weight of up to about 70 kDa across an epithelial cell monolayer.

In addition to assessing the ability of the exemplary permeation enhancing lipids to mediate FD permeation, a MTT assay was performed to determine the effect POVPC; PGPC; Azelaoyl PAF (C16-09:0); glucosyl-sphingosine; 1-O-octadecyl-2-O-methyl-sn-glycero-3-phosphocholine; 16:0-09:0(COOH) phosphocholine and 16:0-09:0(ALDO) phosphocholine have on cell viability. The same negative and positive controls that were used in the FD permeation assay were used in the MTT assay. In all instances, the exemplary permeation enhancing lipids of the present invention had MTT levels comparable to that of the PBS negative control indicating that these lipids did not adversely affect cell viability of the epithelial cell monolayer.

The lipids C16-02:0 PC (C16 PAF), C18-02:0 PC (C18 PAF), C16 Enantiomeric PAF, POVPC, C16-04:1 PC were further characterized by assessing the effect these lipids had on TER and LDH levels with the EpiAirway model system while in the presence of FD molecules with a molecular weight range of 3 kD to 500 kD. The results are summarized in Table 6 below. TER reduction is expressed as the percent of the original TER value at time zero, thus a lower percent TER value equates to a greater TER reduction.

TABLE 6
Percent TER and LDH of an Epithelia in the Presence of
Lipids with Different Molecular Weight FITC-Dextran Molecules

| | | | | % Relative |
|---|---|---|---|---|
| | | | Mean | Cytotoxic |
| | Lipid Name or | FITC- | % of Original | Effect |
| | Control | Dextran MW | TER Value | (LDH) |
| Negative | PBS | FD3 | 88% | 4% |
| Control | | FD10 | 80% | 4% |
| | | FD40 | 83% | 3% |
| | | FD70 | 71% | 3% |
| | | FD500 | 90% | 3% |
| Positive | 0.3% Triton- | FD3 | ND | 99% |
| Control | X100 ™ | | | |
| Lipids | POVPC | FD3 | 7% | 19% |
| | | FD10 | 6% | 11% |
| | | FD40 | 8% | 13% |
| | | FD70 | 8% | 13% |
| | | FD500 | 6% | 12% |
| | C16-02:0 PC | FD3 | 4% | 32% |
| | (C16 PAF) | FD10 | 3% | 35% |
| | | FD40 | 3% | 31% |
| | | FD70 | 3% | 35% |
| | | FD500 | 3% | 26% |
| | C18-02:0 PC | FD3 | 2% | 33% |
| | (C18 PAF) | FD10 | 1% | 33% |
| | | FD40 | 0% | 32% |
| | | FD70 | 0% | 33% |
| | | FD500 | 0% | 26% |
| | C16-04:1 PC | FD3 | 2% | 30% |
| | | FD10 | 2% | 33% |
| | | FD40 | 1% | 28% |
| | | FD70 | 1% | 33% |
| | | FD500 | 0% | 24% |
| | C16 | FD3 | 3% | 34% |
| | Enantiomeric | FD10 | 2% | 32% |
| | PAF | FD40 | 3% | 36% |
| | | FD70 | 2% | 31% |
| | | FD500 | 2% | 33% |

As expected, the negative control PBS failed to reduce TER and did not induce a cytotoxic effect with the low molecular weight or high molecular weight FD molecules. The positive control Triton-X100™ induced high levels of LDH, as expected. In all instances, the permeation enhancing lipids reduced TER to 8% or less of the original TER value of the cells absent any treatment. Further, none of the permeation enhancing lipids induced LDH levels above 35% indicating that the permeation enhancing lipids in the presence of low and high molecular weight molecules do not induce cytotoxicity.

These data show the surprising and unexpected discovery that the exemplary permeation enhancing lipids of the present invention enhance the permeation of both low and high molecular weight molecules across an epithelial cell monolayer without adversely effecting cell viability.

### Example 6

### Permeation Enhancing Lipids Enhance the Permeation of Peptide YY (PYY) and Insulin Across an Epithelial Cell Layer

The present example demonstrates that the exemplary permeation enhancing lipids of the present invention enhance permeation of a biological agent across an epithelial cell monolayer. The data presented in prior Examples of the instant application indicated that the exemplary permeation enhancing lipids of the present invention enhance the permeation of FD across an epithelial monolayer. In the instant example, the ability of permeation enhancing lipids to enhance the permeation of the biological agent, peptide YY (PYY; molecular weight of 3.7 kDa) across the epithelial cell monolayer model system (EpiAirway™) was measured. Also, the efficacy of a permeation enhancing lipid to enhance the permeation of insulin across and epithelial cell layer was measured. Refer to Example 2 of the instant application for general protocol details. Table 7 below shows PYY permeation and TER reduction (% Original TER), cell viability and cytotoxicity results for the lipids, PGPC, C16 PAF, C18 PAF, and PAF-antagonist and glucosyl sphingosine, and the positive control PN159 (delivery peptide) and the negative control, 0.75× PBS in the presence of PYY.

TABLE 7
PYY Permeation, TER Reduction, Cell Viability and Cytotoxicity Results

| | | % PYY | % Original | % Cell | % |
|---|---|---|---|---|---|
| | Sample | Permeation | TER | Viability | Cytotoxicity |
| Lipids | PGPC 500 μM/ | 0.13% | 81% | 113% | 2% |
| | PYY 13.67 mg/mL | | | | |
| | (High) | | | | |
| | C16 PAF/PYY | 3.3% | 1% | ND | 23% |
| | 10 mg/mL | | | | |
| | C18 PAF/PYY | 5.4% | 0.5% | ND | 19% |
| | 10 mg/mL | | | | |
| | PAF-antangonist | 2.4% | 2% | ND | 15% |
| | PAF/PYY | | | | |
| | 10 mg/mL | | | | |
| | Glucosyl | 1.17% | 12% | 109% | 19% |
| | Sphingosine | | | | |
| | 500 μM/PYY | | | | |
| | 13.67 mg/mL | | | | |
| | (High) | | | | |
| Positive | PN159 25 μM/ | 3.71% | 10% | 89% | 33% |
| Controls | PYY 13.67 mg/mL | | | | |
| | (High) | | | | |
| | Special Sauce | 4.7% | 2% | ND | 22% |
| | (in citrate) | | | | |
| Negative | 0.75x PBS/PYY | 0.15% | 67% | 94% | 0% |
| Controls | 13.67 mg/mL | | | | |
| | (High) | | | | |
| | Citrate Buffer | 0.6% | 100% | ND | 3% |

The data in Table 7 indicate that the permeation enhancing lipids in the presence of PYY do not reduce cell viability and/or have minimal effect on cytotoxicity relative to the positive controls PN159 or Special Sauce and the negative controls PBS and citrate buffer. PGPC in the presence of PYY shows limited ability to reduce TER while glucosyl sphingosine in the presence of PYY reduced TER to levels equivalent of PN159 (positive control). However, the permeation enhancing lipids C16 PAF, C18 PAF and PAF-antagonist reduced TER below that of the positive control PN159 and equivalent to the positive control Special Sauce. Further, these permeation enhancing lipids enhanced permeation of PYY equivalent to or above the positive control PN159. Specifically, the PAF lipid C18 PAF enhanced PYY permeation to above 5%, which exceeded any of the positive controls.

The lipid C16 PAF at 1000 μM enhanced the permeation of insulin across the epithelial cell monolayer model system to more than about 3%.

These data show the surprising and unexpected discovery that the exemplary permeation enhancing lipids C16 PAF, C18 PAF, PAF-antagonist and PGPC of the present invention enhance the permeation of a peptide or protein across and epithelial cell layer.

### Example 7

### Permeation Kinetics of Permeation Enhancing Lipids Combined with Excipients

The present example demonstrates that low molecular weight excipients enhance the efficacy of the exemplary permeation enhancing lipids of the present invention to reduce TER and promote the permeation of a FITC-dextran molecular weight 3000 (FD3) and a biological agent, for example insulin across an epithelial cell layer without inducing cytotoxicity. The ability of the permeation enhancing lipids C16 PAF, C18 PAF, C16 Enantiomeric PAF, C16-04:1 PC and POVPC at 1000 μM concentration in the presence of two different buffers, Buffer I (10 mM citrate, pH 5.0; 25 mM lactose; 100 mM sorbitol and 3.4 mM EDTA) and Buffer II (10 mM citrate, pH 5.0; 25 mM lactose; 100 mM sorbitol; 3.4 mM EDTA and 45 mg/ml M-β-CD) to reduce TER and enhance the permeation of FD3 across a monolayer of human-derived tracheal/bronchial epithelial cells (EpiAirway™ Model System) without inducing cytotoxicity (LDH levels) was measured. Also, measured was TER recovery at zero hour and 16 hours post-treatment. Table 8 below shows the permeation enhancing lipids, the concentration at which each lipid was assayed, the buffer used and resulting percent original TER (% original TER), percent apical LDH release (% cytotoxicity), percent FD3 permeation (% FD permeation) and TER recovery in ohms at zero and 16 hours post-treatment. PBS served as a negative control while Special Sauce (described above) and Triton-X100™ served as positive controls.

TABLE 8
Permeation Kinetics of Permeation Enhancing Lipids with Buffers I and II

| | | | | | | TER Recovery | |
|---|---|---|---|---|---|---|---|
| | | | | | | (ohms) | |
| | | | % Original | % | % FD3 | 0 | 16 |
| Treatment | Conc. | Buffer | TER | Cytotoxicity | Permeation | Hour | Hours |
| PBS | N/A | N/A | 98% | 1% | 1% | 512 | 532 |
| Buffer I | N/A | N/A | 9% | 8% | 11% | 45 | 480 |
| Buffer II | N/A | N/A | 14% | 12% | 14% | ND | ND |
| Special | N/A | N/A | 9% | 24% | 20% | 49 | 665 |
| Sauce | | | | | | | |
| Triton- | 0.3% | N/A | ND | 100% | ND | ND | ND |
| X100 ™ | | | | | | | |
| C16 PAF | 1000 μM | I | 1% | 43% | 41% | 9 | 650 |
| | | II | 8% | 13% | 27% | ND | ND |
| C18 PAF | 1000 μM | I | 1% | 33% | 42% | 6 | 497 |
| | | II | 7% | 17% | 27% | ND | ND |
| C16 | 1000 μM | I | 2% | 34% | 46% | 9 | 462 |
| Enantiomeric | | II | 9% | 14% | 23% | ND | ND |
| PAF | | | | | | | |
| C16-04:1PC | 1000 μM | I | 2% | 32% | 44% | 10 | 520 |
| | | II | 23% | 14% | 8% | ND | ND |
| POVPC | 1000 μM | I | 25% | 15% | 53% | 71 | 508 |
| | | II | 17% | 15% | 9% | ND | ND |

ND = no data

The data in in Table 8 show that the excipients lactose, sorbitol and EDTA (Buffer I) enhance the ability of the exemplary lipids C16 PAF, C18 PAF, C16 Enantiomeric PAF, C16-04:1 PC and POVPC of the present invention to promote the permeation of a low molecular weight agent, FD3 (compare to FD3 permeation in Tables 4 and 5). Measured LDH levels indicate that Buffer I does not induce significant cytotoxicity. Further, TER recovery results suggest that epithelial cells incubated with C16 PAF, C18 PAF, C16 Enantiomeric PAF, C16-04:1 PC or POVPC in the presence of Buffer I recover to PBS control levels within 16 hours, indicating the permeation enhancedment induced by the lipids in the presence of Buffer I is reversible. The addition of M-β-CD to the buffer (Buffer II) did not enhance the lipid's ability to enhance permeation of FD3.

Based on the FD3 permeation data in Table 8, the ability of the C16 PAF and C16 Enantiomeric PAF in the presence of Buffers I and II to enhance permeation of the biological agent insulin was assayed. Each lipid was tested at a 1000 μM concentration. Table 9 below shows the insulin permeation results.

TABLE 9
Lipids with Low Molecular Weight Excipients Mediate Insulin Permeation

| | | | % Insulin |
|---|---|---|---|
| Treatment | Concentration. | Buffer | Permeation |
| PBS | N/A | N/A | 0% |
| Buffer I | N/A | N/A | 1% |
| Special | N/A | N/A | 4% |
| Sauce | | | |
| C16 PAF | 1000 μM | I | 4% |
| | | II | 8% |
| | | PBS | 3% |
| C16 | 1000 μM | I | 3% |
| Enantiomeric | | II | 8% |
| PAF | | | |

The data in Table 9 shows that the lipids C16 PAF and C16 enantiomeric PAF enhance the permeation of insulin across an epithelial cell monolayer in the presence of Buffer I and Buffer II. Specifically, the lipids in the presence of Buffer II enhance insulin permeation to a greater degree than Buffer I. Taken together with the data from Table 8, the permeation enhancing effects of Buffer I and Buffer II appear to be biological agent dependent.

### Example 8

### Chemical Structures of Exemplarv Permeation Enhancing Lipids

The present example illustrates the chemical structure of exemplary permeation enhancing lipids of the present invention.

The chemical structure of the exemplary permeation enhancing lipid 1-palmitoyl-2-(5′-oxo-valeroyl)-sn-glycero-3-phosphocholine (POVPC) is as follows:

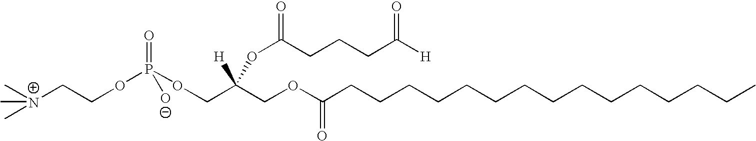

The chemical structure of the exemplary permeation enhancing lipid 1-palmitoyl-2-glutaroyl-sn-glycero-3-phosphocholine (PGPC) is as follows:

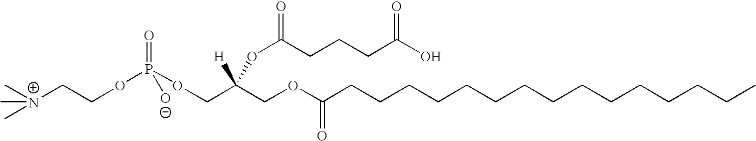

The chemical structure of the exemplary permeation enhancing lipid 1-O-hexadecyl-2-azelaoyl-sn-glycero-3-phosphocholine (C16-09:0) is as follows:

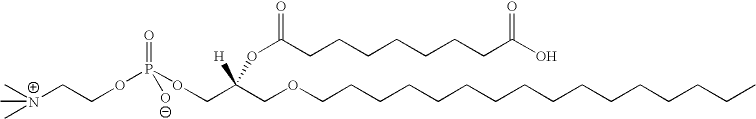

The chemical structure of the exemplary permeation enhancing lipid 1-palmitoyl-2-azelaoyl-sn-glycero-3-phosphocholine (16:0-09:0(COOH)PC) is as follows:

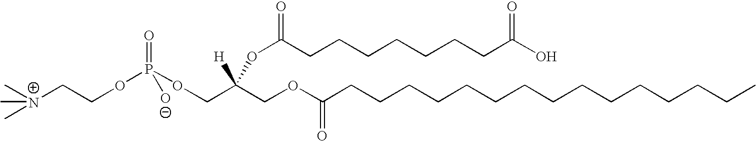

The chemical structure of the exemplary permeation enhancing lipid 1-palmitoyl-2-(9′-oxo-nonanoyl)-sn-glycero-3-phosphocholine (16:0-09:0(ALDO)PC) is as follows:

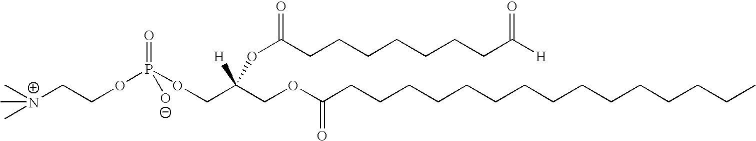

The chemical structure of the exemplary permeation enhancing lipid 1-O-Octadecyl-2-O-Methyl-sn-Glycero-3-Phosphocholine (18:0-1:0 Diether PC) is as follows:

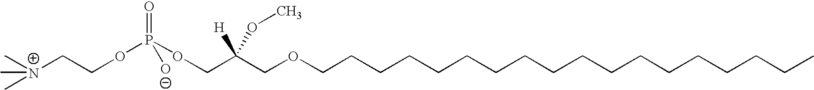

The chemical structure of the exemplary permeation enhancing lipid 1-O-Hexadecyl-2-Azelaoyl-sn-Glycero-3-Phosphocholine (Azelaoyl PAF) is as follows:

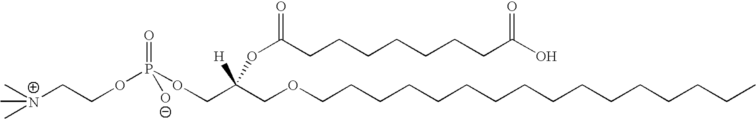

The chemical structure of the exemplary permeation enhancing lipid 1-O-Hexadecyl-2-Hydroxy-sn-Glycero-3-Phosphocholine (C16 Lyso-PAF) is as follows:

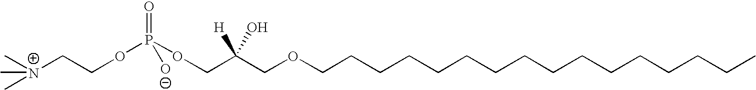

The chemical structure of the exemplary permeation enhancing lipid 1-O-Octadecyl-2-hydroxy-sn-Glycero-3-Phosphocholine (C18 Lyso-PAF) is as follows:

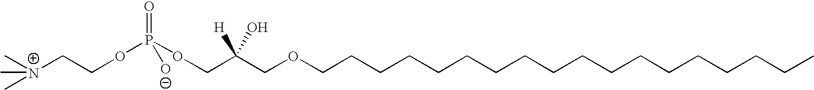

The chemical structure of the exemplary permeation enhancing lipid 1-O-Octadecyl-2-Acetoyl-sn-Glycero-3-Phosphocholine (C18-02:0 PC(C18 PAF)) is as follows:

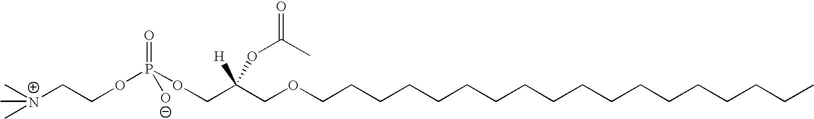

The chemical structure of the exemplary permeation enhancing lipid 1-O-Hexadecyl-2-Butenoyl-sn-Glycero-3-Phosphocholine (C16-04:1 PC) is as follows:

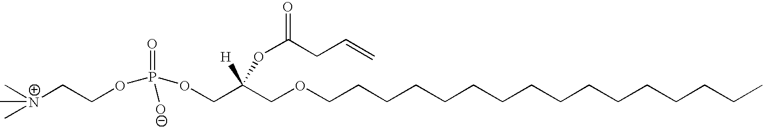

The chemical structure of the exemplary permeation enhancing lipid 1-O-Hexadecyl-2-Butyroyl-sn-Glycero-3-Phosphocholine (C16-04:0 PC) is as follows:

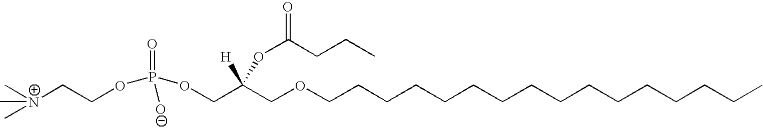

The chemical structure of the exemplary permeation enhancing lipid 3-O-Hexadecyl-2-Acetoyl-sn-Glycero-1-Phosphocholine (C16 Enantiomeric PAF) is as follows:

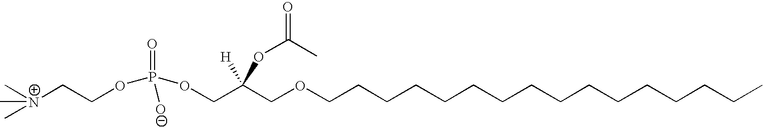

The chemical structure of the exemplary permeation enhancing lipid 1-Palmitoyl-2-Acetoyl-sn-Glycero-3-Phosphocholine (16:0-02:0 PC) is as follows:

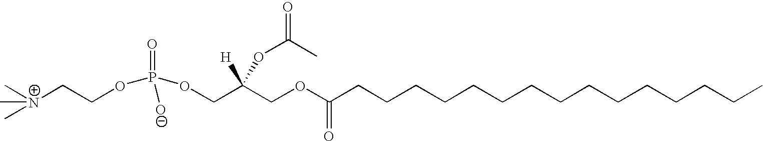

The chemical structure of the exemplary permeation enhancing lipid 1-O-Hexadecyl-2-Acetoyl-sn-Glycero-3-Phosphocholine (C16-02:0 PC(C16 PAF)) is as follows:

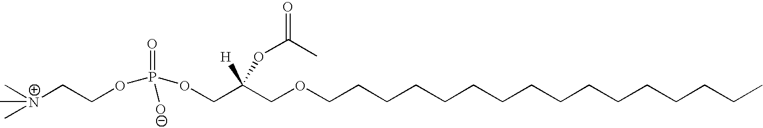

What is claimed is:

1. A composition comprising a biologically active agent and a permeation enhancing lipid, wherein the permeation enhancing lipid is a platelet activating factor antagonist or a biologically inactive platelet activating factor, and increases permeability of the biologically active agent across a tissue layer.

2. The composition of claim 1, wherein the permeation enhancing lipid is selected from the group consisting of 1-O-alkyl-2-hydroxy-sn-glycero-3-phosphocholine, 3-O-alkyl-2-acetoyl-sn-glycero-1-phosphocholine and 1-O-alkyl-2-O-acetyl-sn-glycero-3-phospho(N,N,N-trimethyl)hexanolamine.

3. The composition of claim 2, wherein the lipid is comprised of a (C₈-C₂₂)alkyl.

4. The composition of claim 1, wherein the permeation enhancing lipid is selected from the group consisting of 1-O-hexadecyl-2-hydroxy-sn-glycero-3-phosphocholine; 1-O-octadecyl-2-hydroxy-sn-glycero-3-phosphocholine; 3-O-hexadecyl-2-acetoyl-sn-glycero-1-phosphocholine and 1-O-hexadecyl-2-O-acetyl-sn-glycero-3-phospho(N,N,N-trimethyl)hexanolamine.

5. The composition of claim 1, wherein the tissue layer consists of mucosal tissue.

6. The composition of claim 5, wherein the mucosal tissue is comprised of epithelial cells.

7. The composition of claim 6, wherein the epithelial cell is selected from the group consisting of tracheal, bronchial, alveolar, nasal, pulmonary, gastrointestinal, epidermal or buccal.

8. The composition of claim 1, wherein the biologically active agent is a peptide or protein.

9. The composition of claim 1, wherein the biologically active agent is between about 1 kiloDalton and about 50 kiloDaltons.

10. The composition of claim 1, wherein the biologically active agent is between about 3 kiloDaltons to about 40 kiloDaltons.

11. The composition of claim 8, wherein the peptide or protein is selected from the groups consisting of peptide YY (PYY), parathyroid hormone (PTH), interferon-alpha (INF-α), interferon-beta (INF-β), interferon-gamma (INF-γ), human growth hormone (hGH), exenatide, glucagon-like peptide-1 (GLP-1), glucagon-like peptide-2 (GLP-2), glucagon-like peptide-1 derivatives, oxytocin, insulin and carbetocin.

12. The composition of claim 1, wherein the composition is further comprised of at least two poloyls.

13. The composition of claim 12, wherein the poloyls are lactose and sorbitol.

14. The composition of claim 1, wherein the composition is further comprised of a chelating agent.

15. The composition of claim 14, wherein the chelating agent is diamine tetraacetic acid (EDTA).

16. The composition of claim 1, wherein the composition is aqueous.

17. The composition of claim 1, wherein the composition is solid.

18. A process of increasing the permeability of a biological agent across a tissue layer comprising contacting the tissue layer with a composition comprising the biological agent and a permeation enhancing lipid, wherein the permeation enhancing lipid is a platelet activating factor antagonist or a biologically inactive platelet activating factor.

19. The process of claim 18, wherein the permeation enhancing lipid is selected from the group consisting of 1-O-alkyl-2-hydroxy-sn-glycero-3-phosphocholine, 3-O-alkyl-2-acetoyl-sn-glycero-1-phosphocholine and 1-O-alkyl-2-O-acetyl-sn-glycero-3-phospho(N,N,N-trimethyl)hexanolamine.

20. The process of claim 19, wherein the lipid is comprised of a (C₈-C₂₂)alkyl.

21. The process of claim 18, wherein the permeation enhancing lipid is selected from the group consisting of 1-O-hexadecyl-2-hydroxy-sn-glycero-3-phosphocholine; 1-O-octadecyl-2-hydroxy-sn-glycero-3-phosphocholine; 3-O-hexadecyl-2-acetoyl-sn-glycero-1-phosphocholine and 1-O-hexadecyl-2-O-acetyl-sn-glycero-3-phospho(N,N,N-trimethyl)hexanolamine.

22. The process of claim 18, wherein the tissue layer consists of mucosal tissue.

23. The process of claim 22, wherein the mucosal tissue is comprised of epithelial cells.

24. The process of claim 23, wherein the epithelial cell is selected from the group consisting of tracheal, bronchial, alveolar, nasal, pulmonary, gastrointestinal, epidermal or buccal.

25. The process of claim 18, wherein the biologically active agent is a peptide or protein.

26. The process of claim 18, wherein the biologically active agent is between about 1 kiloDalton and about 50 kiloDaltons.

27. The process of claim 18, wherein the biologically active agent is between about 3 kiloDaltons and about 40 kiloDaltons.

28. The process of claim 25, wherein the peptide or protein is selected from the groups consisting of peptide YY (PYY), parathyroid hormone (PTH), interferon-alpha (INF-α), interferon-beta (INF-β), interferon-gamma (INF-γ), human growth hormone (hGH), exenatide, glucagon-like peptide-1 (GLP-1), glucagon-like peptide-2 (GLP-2), glucagon-like peptide-1 derivatives, oxytocin, insulin and carbetocin.

29. The process of claim 18, wherein the composition is further comprised of at least two poloyls.

30. The process of claim 29, wherein the poloyls are lactose and sorbitol.

31. The process of claim 18, wherein the composition is further comprised of a chelating agent.

32. The process of claim 31, wherein the chelating agent is diamine tetraacetic acid (EDTA).

33. The process of claim 18, wherein the composition is aqueous.

34. The process of claim 18, wherein the composition is solid.
